(19) United States
(12) Patent Application Publication
(10) Pub. No.: US 2003/0059933 A1
(43) Pub. Date: Mar. 27, 2003

(54) Bioartificial device for propagation of tissue, preparation and uses thereof

(76) Inventors: Patrick A. Tresco, Sandy, UT (US); Roy Biran, Holladay, UT (US); Mark D. Noble, Brighton, NY (US)

Correspondence Address: David A. Jackson, KLAUBER & JACKSON, 411 Hackensack Street, 4th Floor, Hackensack, NJ, 07601, US

(21) Appl. No.: 10/075,129

(22) Filed: Feb. 11, 2002

Related U.S. Application Data
(63) Continuation of application No. PCT/US00/21931, filed on Aug. 10, 2000.
(60) Provisional application No. 60/148,160, filed on Aug. 10, 1999.

(51) Int. Cl. A01H 1/00; C12M 1/14; C12M 3/04

(52) U.S. Cl. 435/299.1; 800/268

(57) ABSTRACT

The present invention relates to a device and corresponding methods for its use, in the propagation of tissue. The device comprises a bioartificial composite comprised of a substrate having at least one surface capable of the reception and growth promoting retention of a cellular preparation, and a first layer of adherent cells disposed on said surface. The first layer is prepared from the cellular preparation, and the cells comprising the first layer have cytoskeletal elements aligned uniformly, so that the bioartificial composite acts as a template to accept a second layer of cells upon the first layer, said second layer comprising an organized layer oriented in the direction of said first layer, wherein said substrate has at least one surface defined by a critical surface curvature and/or topography. The device may be implanted for the promotion of tissue regrowth, or may be used to develop tissue on an ex vivo basis, for implantation or for experimentation.

### GOVERNMENTAL SUPPORT

The research leading to the present invention was supported, at least in part, by ATP Cooperative Agreement Grant No. 97-07-0028. Accordingly, the Government may have certain rights in the invention.

### FIELD OF THE INVENTION

This invention relates generally to devices for the preparation and delivery of tissue corresponding to that found in mammals, to effect growth, regrowth, or repair of tissues damaged or destroyed by disease, accident or surgery. The invention is particularly useful for the preparation of implants of multiple cell layers, and to promote regrowth in vivo. The invention also relates to uses of the devices for e.g. the growth of multifilamentous tissue such as nerve tissue, and can also be used for the delivery of drugs, hormones and other factors to sites in a host, as well as for gene therapy, and in identifying, assaying or screening of cell-cell interactions, lineage commitment, development genes and growth or differentiation factors. Additionally, the present invention relates to methods and devices used for the creation of multiple layers of cells that are directionally aligned and to the application of such to the treatment of diseases, disorders or deficiencies resulting from the loss of tissue function, metabolic or endocrine in nature.

### BACKGROUND OF THE INVENTION

The invention relates to methods of generating multiple layers of cells that are oriented in the same direction and their use as devices for therapeutic purposes.

One of the most striking features of virtually all normal tissues is the high degree of order and patterning that occurs during all stages of development. Whether one examines such diverse processes as the orderly formation of axon tracts, the creation of arrays of skeletal muscle fibers or the formation of kidney glomeruli, it is clear that the creation of normal tissue follows precise rules of organization.

The creation of a single layer of ordered cells would be of little value unless the order is successfully transmitted from one cell layer to the next. Thus, the creation of order in a multilayered structure is a necessary aspect of normal tissue development. Little is known about how such order is generated in the body nor have techniques been developed that induce in a controllable manner the generation of multilayered ordered tissue structures in vitro or in vivo.

Just as order is a striking feature of normal tissues, so is disorder a feature of pathology. Disorder is seen in degenerative processes and is also seen in the failure of regeneration to create fully normal tissue. One common example of such disorder in tissue regeneration is seen in scar tissue in which the precisely patterned organization of cells that existed prior to injury is not reformed. On the surface of the body, such scarring can be disfiguring. When it occurs in deeper structures, function can be severely compromised. For example, the disordered organization of scarring following surgery can result in weakened tissue, scarring within a regenerating kidney or liver can impair normal function; and in the nervous system, scarring after injury can prevent normal regenerative processes. Indeed, the generation of ordered structures is so essential to the creation of a functioning nervous system that not even simple reflex loops can be established in its absence let alone the complexity of higher order motor and cognitive processes.

Even the most cursory examination of the simplest tissues of the body demonstrate that order is not simply a property of single monolayers of cells. Instead, order seen in the body reflects the ability of multiple cellular layers to become organized in an integrated and well-defined manner.

If disorder is a feature of pathology and order is a feature of normal tissue function and if normal tissue function requires the transmission of order through multiple layers of cells, then it follows that a critical goal in the field of tissue repair is the discovery of means of creating structures that display order through multiple layers of cells as a result of intentional design features utilized by the practicioner of the art. In order for such discoveries to have maximal opportunities to be utilized in tissue repair, it would be beneficial if the design features could be readily applied in such a manner as to allow large scale production at relatively low cost and with the ability to vary design features over a wide range to allow for introduction of specific features for specific applications. Thus, two goals of the invention are to provide means of creating multi-layered organized structures of cells and also to provide means of creating such structures in a flexible manner that can be applied at low cost and with great reproducibility.

The failure to achieve the above two goals is shown clearly by examples from the very fields that are most closely related to the purposes of the invention. These are the fields of tissue repair by cell transplantation and the study of topographic influences on cell behavior.

The field of cell transplantation is typified by one of two approaches. One is the transplantation of encapsulated cells that have no physical contact with the host environment and the second is the transplantation of cells that are able to integrate into the host tissue. The latter approach is being pursued with regards to the repair of many different kinds of tissues and the general strategy applied is the same in all cases, namely, to inject or transplant a bolus of dissociated cells into a specific region to be repaired and hope that the host environment and/or properties of the transplanted cells will be sufficient to confer order.

Examples of the transplantation of cells as individual entities free to integrate into host tissue can be provided from a number of different tissues but the principles are the same in all cases. The application of this procedure to repair of CNS damage is discussed as a non-limiting example of the general class of problem that underlies this approach.

Demonstrations that precursor cells injected into the damaged CNS will integrate into normal tissue, forming new myelin or new neuronal circuits has created hopes that similar approaches can be used in the effective treatment of human diseases. While the extent of repair that can be associated with such cell injection as for example into the damaged nervous system offers great hope for reconstitution of dysfunctional CNS tissue, it seems unlikely, however, that existing approaches in this nascent field represent an optimal approach to restoring order or directionality to damaged circuitry. This is because following cell injection, the newly generated neurons are still required to extend axons to target organs in a precisely ordered way. This requires growth of axons to the proper location often through regions of damaged CNS tissue that in-and-of itself displays disorder. It is readily apparent, from micrographs of the growth patterns of transplanted cells, that neuronal growth patterns frequently do not exhibit the precisely ordered growth and directionality of their normal endogenous counterparts.

If the transplantation of dissociated cells is insufficient to create order, then it is necessary to discover how one might intentionally confer order on such cells. In light of the previously discussed importance of transmission of order through multiple cellular layers, it can further be stipulated that it is necessary to discover means of intentionally conferring order in structures consisting of multiple layers of cells.

The means by which order is created has been the subject of extensive investigation and people have invoked gradients of growth factors, attractive and repulsive haptotactic or substrate bound signals and topographic features. Despite extensive investigation, however, no publications have disclosed a means of creating intentionally ordered structures involving multiple cellular layers.

It has been known for many years that it is possible to impose order on small numbers of cells growing in single layers on a variety of substrates. In particular, surface topography can modulate shape, orientation and adhesion of many—and perhaps all—types of cells (Brunette, 1986a; Curtis and Clark, 1990; Dunn and Brown, 1986). Curtis and Clark, in particular (1990) noted that all cells growing on a substratum must contend with topography and drew attention to the potential importance of the reactions of cells to topographic features in vivo for morphogenesis, cell invasion, repair and regeneration. Several studies have shown that microfilament bundles (Dunn and Heath, 1976; Ben-Ze'ev, 1986), focal contacts (O'Hara and Buck, 1979) and microtubules (Oakley and Brunette, 1992) align with topographic features such as grooves. Cell shape also can be markedly influenced by surface topography (Oakley and Brunette, 1993; Dunn and Brown, 1986; Curtis and Clark, 1990), as can cell growth (Watt, 1987; Folkman and Moscona, 1978; cytoskeleton gene expression (Web et al., 1989), extracellular matrix metabolism (Watt, 1986; McDonald, 1989) and cell differentiation.

It is also clear that a topographic feature that has little effect on cells when presented as a single instance can modulate cell behavior when presented as a closely spaced multiple array. Such a result is consistent with that suggested by a number of in vivo studies of animal development (Bard and Higginson, 1977; Lofberg and Ahlfors, 1978; Lofberg et al., 1980; Nakatsuji and Johnson, 1984; Newgreen, 1989; Wood and Thorogood, 1984, 1987). For example, mesenchymal cells of the developing teleost fin bud are believed to be contact guided by collagen actinotrichia forming a double layer of ridge substratum through which they migrate into cell-free space (Wood and Thorogood, 1984, 1987) and were found to be contact guided in a similar manner by artificially grooved substrata (Wood, 1988). In addition, oriented extracellular matrix material is though to influence cell shape and locomotion in vivo, for example, in the orientation of fibroblasts during corneal development (Bard and Higginson, 1977), mesoderm migration during gastrulation (Nakatsuji and Johnson, 1984) and in early neural crest cell migration in the axolotl (Lofberg and Ahlfors, 1978; Lofberg et al., 1980) and quail (Newgreen, 1989).

Attempts to define the principles that underlie contact guidance of cells has been heavily dependent on the generation of artificial surfaces with specific topographic features. Early work on contact guidance (Weiss, 1945, 1958) showed that cells aligned and migrated along fibers and grooves. Later work (Curtis & Varde, 1964) suggested that cells were probably reacting to topographical features rather than to any molecular orientation.

The use of micro-fabrication techniques to create surfaces with regular and repeating features has been applied by several groups to analyze cell behavior in response to topographically defined surfaces (e.g., Brunette, 1986; Brunette, Kenner Gould, 1983; Dunn & Brown, 1986). For example, in a study by Clark et al. (Clark, P.; Connolly, P.; Curtis, A. S. G.; Dow, J. A. T.; and Wilkinson, C. D. W. (1987) Topographical Control of Cell Behavior. I. Simple Step Cues., Development, 99:439-448.) the growth surface for cells was patterned by photolithography. BHK cells were first studied and their ability to cross a single step was found to be dependent on step height. If a cell went from a lower to a higher step, even of only one micron in height, then all steps decreased crossing. In contrast, if the cell went from a high step to a lower one, it was found that a one micron step had no effect, but a three micron step inhibited crossing. In contrast, all step heights increased the degree of alignment of the cells studies. These researchers also examined the effect of the surface topography under study on cells derived from chick embryo hemispheres and judged to be neurons on the basis of their morphology and found similar effects. In general, the effect of increasing step height on a cell's ability to cross was a gradual one was modified by the adhesive properties of the substrate and the effects were probabilistic in nature rather than being absolute at a particular step height.

For example, European Patent Application EP84308230.6 discloses the location of biological cells in a predetermined spatial disposition on a solid nonbiological substrate, by providing the substrate with a plurality of surface discontinuities defining cell adhesion enhanced and/or cell-adhesion orienting zones, for example grooves or ridges. However, it does not address the concept of inducing the formation of multilayered tissue structures, either ex vivo or in vivo. More recently, the microtopographical control of cell behavior by the use of a grooved substrate has been described by Clark et al,; Development 108; 635-444 (1990), however this representative reference is likewise silent as to the preparation of multiple layer tissue structures as is envisioned herein.

Identification of which regular and repeating topographic features are the most important in determining cellular behavior has focused attention on ridge width, grating period and groove depth. For example, Dunn and Brown (Dunn, G. A. and Brown, A. F. (1986) Alignment of Fibroblasts on Grooved Surfaces described by A Simple Geometric Transformation, J. Cell Sci., 83:313-340) used extensive mathematical analysis to determine that ridge width is the main parameter affecting cell alignment with alignment being inversely proportional to ridge width. Groove width was found to have a small additional effect. Still other studies have shown that decreasing the grating period and increasing the depth of microgrooves increased alignment with depth being dominant in its effects (Clark et al., 1990). In these studies (Clark, P.; Connolly, P.; Curtis, A. S. G.; Dow, J. A. T.; and Wilkinson, C. D. W. (1990) Topographical Control of Cell Behavior: II. Multiple Grooved Substrata, Development, 108:635-644.), the authors examined growth of BHK cells, MDCK cells and chick embryo cerebral neurons on grooved substrata of dimensions varying from 4-24 micron repeat, 0.2-1.9 micron depth. Alignment was inversely proportional to spacing (intergroove distance), but this feature was much less important than groove depth. In addition, not all cells were effected in the same manner by a particular topography. For example, BHK cells interacted with the surface as single units while the response of MDCK cells depended on whether or not the cells were isolated or part of an epithelial cell island as well as on the depth of the grooves. If the grooves had a depth greater than 0.56 microns, however, then colonies became elongated. For cells derived from chick embryo hemispheres and which had a neuronal morphology, the outgrowth of putative neurites appeared unaffected on the one micron patterns. On two micron deep patterns, in contrast, neurite outgrowth was markedly aligned to groove direction. Similar differences between different cell types were also seen in further studies by Clark et al. (1991) in which grating patterns of different depths with submicrometer periods (260 nm period; 130 nm grooves; 130 nm separation) were created through use of a laser interferometer and reactive ion etching on fused quartz surfaces. BHK cells readily aligned with these grooves with the degree of alignment being dependent on groove depth. Single MDCK cells also aligned but failed to do so when they were in epithelial colonies. Patterns of outgrowth of neurites from chick embryo neurons was not affected by these grating surfaces.

Although neuronal outgrowth patterns were not affected by growth on the ultrafine surfaces discussed in studies by Clark et al. (1991), there is nonetheless some evidence that growth cones of neurites are susceptible to topographic guidance by single steps (Clark et al., 1987) larger grooved substrata (Clark et al., 1990), grating structures (Hirono et al., (1988) and aligned fibrillar structures (Ebendal, 1976, 1977). For example, Hirono et al., (1988) examined the behavior of spinal ganglion neurons derived from adult rodents and grown on glass plates on which grating-associated microstructures were fabricated with lithographic techniques. The grooves created a striking bidirectional growth of the nerve fibers. The extent of alignment of nerve fibers was sensitive to both width and depth of the microgrooves (which varied from 0.1-10 microns). The authors also observed a highly significant reduction in the number of branchings counted in a single length of neurite. Although they only looked at 5 cells in each experimental group, they reported a reduction from 27+/−2.7 branches per 1 mm length of neurite to 7.9+/3.3 branchings per 1 mm length of neurite when growth was compared on non-grooved substrates, respectively. After 48 hours of growth, the total length of neurites ranged from 1800-5600 microns (average=3700 microns) on the microstructures and from 1300-3700 (2720) microns average length) without microstructures, growth rates of 1.86 mm/day and 1.36 mm/day, respectively. As for other investigators, these authors concluded that the recognition of the microstructures by the neurites and growth cones was almost exclusively mechanical.

It is striking that in all of the above publications no information is provided on the crucial problem of how to create order through multiple cellular layers. It is even more striking that in light of the long history of interest in understanding how to develop monolayers of patterned cells and the long history of knowledge regarding normal tissue structure that there is nothing present in the art that teaches the practitioner of the art how to create patterning that is transmitted from one layer of cells to the next in order to create a patterned multilayered structure.

Unless otherwise defined, all technical and scientific terms have the same meaning as commonly understood by one of ordinary skill in the art to which this invention belongs. Although other materials and methods that are similar or equivalent to those mentioned can be used in the utilization or testing of the present invention, the preferred methods are described below. All publications, patent applications, patents and other reference material mentioned are incorporated by the reference. In addition, the materials, methods and examples are only illustrative and are not intended to be limiting.

The citation of references herein shall not be construed as an admission that such is prior art to the present invention.

### SUMMARY OF THE INVENTION

In accordance with a first aspect of the present invention, there are provided devices and methods of the present invention attain the creation of oriented cell growth and morphological arrangement that extends through more than one cell layer. Particularly, the present invention for the first time identifies a composite structure that promotes regulated multi-layer cell proliferation that corresponds to the structure of living tissue and thereby facilitates prosthetic and regenerative procedures and strategies heretofore not possible. While the present invention has wide applications, it is particularly suitable as a therapeutic treatment to repair, augment or restore function of diseased, damaged or genetically dysfunctional tissue through the transplantation into specific sites in the body, such as the repair of central or peripheral nervous tissue, tendon or muscle. The device is also particularly suitable for transplanting genetically engineered cells to be used for the regulated delivery of a desired therapeutic molecule and can be used as a cell culture device for basic research.

Accordingly, the invention covers a device for the propagation of tissue comprising a bioartificial composite comprised of a substrate having at least one surface capable of the reception and growth promoting retention of a cellular preparation, and a first layer of adherent cells disposed on said surface. The first layer is prepared from the cellular preparation, and the cells comprising the first layer have cytoskeletal elements aligned uniformly, so that the bioartificial composite acts as a template to accept a second layer of cells upon the first layer, said second layer comprising an organized layer oriented in the direction of said first layer, wherein said substrate has at least one surface defined by a critical surface curvature and/or topography.

In its simplest embodiment, the device of the present invention comprises a composite of a substrate for the attachment of anchorage-dependent cells which contains non-uniform grooved axially aligned surface to topography coated with suitable cell attachment molecules; and a first layer of cells attached to the substrate which first layer undergoes morphological rearrangement to align its morphology with the pattern of the underlying surface topography. As illustrated in FIG. 1, infra., the device thus constituted is adapted to receive the addition of another cell layer that attaches to the upper surface of the first adherent cell layer and also rearranges to align with the underlying substrate features.

More particularly, the device may be used for the propagation of tissue such as for experimentation, or for implantation as described in detail hereinafter. Also, the substrate of the device of the invention has at least one cell accepting surface defined by an oriented surface roughness of at least 200 nm root mean squared. Also, the substrate preferably has at least one cell accepting surface defined by a surface curvature of equal or greater than 0.016 microns⁻¹, and may define a repeating surface structure.

The devices of the present invention may be planar in overall configuration, such as strips or sheets, or may be filamentous, fibrous or cylindrical. The critical aspect of the devices is their topography and the concomitant ability to promote and achieve oriented cell growth through multiple layers. In this last mentioned connection, the devices of the invention may include and constitute tissues developed by the sequential contiguous growth of different cell types upon each other. For example, a layer of neurons may be grown directly over a layer of glial cells and may thereby replicate living neural tissue.

In a further aspect of the invention, the substrate of the devices may be coated with a biocompatible, growth promoting preparation which preparation minimizes non-specific protein binding and optimizes attachment of the cells of the first layer. Suitable materials for the preparation include and are selected from the group consisting of surfactants, cell adhesion molecules, polycations, cell growth factors, and mixtures thereof.

As described earlier, the devices of the invention may be planar, filamentous or cylindrical, among various shapes. As will be discussed later on herein, the filamentous variety comprehends single as well as multiple filaments, as would be the case in the preparation of a nerve bundle or a branched structure. In the case where the bioartificial composite is defined by at least one and possibly multiple cylindrical substrates, such a multiple structure is attained.

With respect to filamentous or cylindrical structures, the device substrate may preferably have a diameter of less than 300 μm. The is significantly smaller than has been considered let alone achieved, in the extant literature, and represents one of the characterizing features hereof.

Also, the substrate of the device of the invention further defines an axially aligned surface topography, and is coated with cell attachment molecules; and a layer of cells attached to said molecules, which cells are adapted to undergo morphological rearrangement to align with the long axis of said substrate. There is also at least one second cell layer of different cells that attached to the free upper surface of the said first layer, which is also adapted to undergo the same said morphological rearrangement.

In one embodiment of the invention, the morphological rearrangement of the said first layer of cells is promoted and effected by the imposition of suitable force on said first layer and/or said substrate. This force can be imposed by eg. stretching of the substrate, or the application of fluid pressure on the surface. The result of the imposition of stress in this fashion will be to promote cell orientation and alignment.

Accordingly, force may create a morphologically arranged layer of cells; this force may be fluid tangential shear where the cells align with the direction of fluid flow or may be uniaxial strain in which the cells align in the direction of substrate strain after the first layer of cells undergoes morphological rearrangement to align its morphology as described and the addition of another cell layer that attached to the upper surface of the first adherent cell layer and also rearranges to align with the long axis of the cylinder.

In a further embodiment of the invention, the device may be prepared by a method that comprises:

a. preparing a suitable biomaterial as a three dimensional structure selected from sheets, strips, strands of indefinite length and fibers;

b. treating at least one outer surface of the biomaterial prepared in Step a. to form thereon at least one said surface for the reception of said first layer of cells;

c. recovering said treated biomaterial defining the said at least one surface of Step b.;

wherein said biomaterial film of Step c. is adapted to serve as substrate for said device.

The substrate so prepared may then be seeded with a cell preparation and incubated to allow the cells to grow to form the first layer and to thereby form the bioartificial composite. In turn, the composite may be implanted in a patient at the location of desired repair, whereby the growth of said tissue takes place in the host. In this manner therefore, the invention comprehends and extends to a method for the preparation of a composite capable of tissue repair by the promotion of tissue regrowth in situ. As described herein, the cellular preparation that is disposed on teh device may be of a different cell type from that of the tissue the regrowth or formation of which is desired or intended. This is described herein with respect to the overlay of glial cells and neurons.

In a further embodiment, the method of the invention extends to the use of a cellular preparation that is genetically modified to deliver a therapeutic compound useful in the treatment of disease or the promotion of tissue repair. In such instance, the device may serve as a sustained release structure, affording ratable, extended treatment to a particular tissue or organ in need of same.

Thus, the invention extends to a method for the preparation of tissue useful for repair of tissues or organs in a host, which method comprises:

a. preparing a substrate defining a surface having the morphological characteristics of the desired tissue;

b. applying to the surface of Step a. cellular preparation, said cellular preparation comprising a quantity of cells capable of growth and aggregation to form said tissue;

c. incubating the substrate of Step b. under conditions promoting the growth of said tissue thereon; and

d. recovering the tissue prepared in Step c.

The tissue thus prepare ex vivo may then be used for tissue repair or reconstruction by implantation or other known techniques.

A further aspect of the invention relates to the preparation of tissue useful for testing, development and discovery, which method may correspond to the method just recited and described. A particular embodiment of such a method is set forth below and comprises:

a. preparing a substrate defining a surface having the following characteristics:

i. at least one cell accepting surface defined by an oriented surface roughness of at least 200 nm root mean squared;

ii. at least one cell accepting surface defined by a surface curvature of equal or greater than 0.016 microns⁻¹; and

iii. said substrate defines a repeating surface structure;

b. applying to the surface of Step a. a cellular preparation, said cellular preparation comprising a quantity of cells capable of growth and aggregation to form a layer of cells;

c. incubating the bioartificial product of Step b. with a different type of cell to effect growth of said tissue thereon; and

d. recovering the tissue prepared in Step c.

The tissue prepared in this manner may be used for therapeutic purposes as described above, or may be used as as a benchtop testing system or tissue surrogate.

In a yet further aspect the invention provides a method of repairing damaged tissue in a patient by providing the device at or adjacent the damage site. The invention includes the disposition of the device at the site and the promotion of the growth thereon of the second and subsequent layers of cells to reform the tissue, or the development of substantial overlay and growth of the second layer of cells of the tissue in object ex vivo followed by the implantation of the resulting device at the site. This latter strategy has applicability to numerous circumstances in which, for example, entire tissue is lost to trauma or removal in an operation. The implant can integrate with the original tissue during the healing process. In any of the scenarios proposed above, the orderly growth of cells is promoted, such that the cellular ordering of the newly formed tissue more closely matches the original cell structuring and function.

In one embodiment, the substrate may be prepared from a biodegradable material which becomes resorbed in vivo and effectively disappears from the site of implantation.

However, in other instances the device may be non-resorbable such as in the case of permanent implants or in the instance where the device is to be used to replace or augment lost or damaged supporting tissue such as bone and the like. Implants including metallic, plastics and ceramic implants are used in connection with joint repair, for example, hip joint prostheses. Such implants may be provided with the cell growth orienting means integrally formed or provided on the surface of the implant itself; or the cell growth orienting means may be on a separate substrate sheet provided on the surface of the implant (such as by wrapping around the implant or adhering thereto). The substrate sheet may be resorbable or non-resorbable.

As an example of an application of any of the devices of the invention is their use to restore function in the damaged nervous tissue in which case the first adherent cell layer would be comprised of one of several forms of glial cell such as astrocyte or schwann cell or a cell genetically modified to behave as a neuronal growth permissive and/or neurotrophic substrate; and the second layer comprised of neurons. The device may be implanted into nervous tissue with both cell layers or with just the first layer with the second layer being provided by the growth of host neurons. Any of the substrates can be used as a therapeutic implant to replace lost tissue function or as a sustained delivery implant to deliver a therapeutic molecule. Similar approaches would be to augment connective, endocrine or nervous tissue.

In a further embodiment, the device of the invention may be used to deliver one or more agents, drugs, hormones or growth factors, by disposing within or upon the first cellular layer, appropriate vesicles or the like containing these agents, that will release them in situ. Particular examples of biologically active molecule which can be delivered by means of implantation with a device of the invention include enzymes for catalyzing the production of non-peptidyl neurotransmitter (e.g., acetylcholine), neurotransmitters, and neurotrophic factors. For example, enzymes can be introduced which increase of the production of needed chemicals, e.g., neurotransmitters or catacholamines in the brain, particularly in the brains of people suffering from neurodegenerative diseases such as Parkinson's disease, Huntington's Disease, and epilepsy. In addition, a variety of neurotrophic factors can be delivered, and examples of such neurotrophic factors include Brain-Derived Neurotrophic Factor (BDNF), Nerve Growth Factor (NGF), Glial-Derived Neurotrophic Factor (GDNF), Neurotrophin-3 (NT-3), Neurotrophin-4 (NT-4), and Villiary Neurotrophic Factor (VNF).

Other objects and advantages will become apparent to those skilled in the art from a review of the following description which proceeds with reference to the following illustrative drawings.

### BRIEF DESCRIPTION OF THE DRAWINGS

FIG. 1 is a combination of slides and AFM profiles illustrating surface topography and showing DRG axonal outgrowth on a bed of perinatal cortical astrocytes. Astrocytes were plated on polypropylene substrates of increasing oriented groove depth. After reaching confluence, DRG neurons were plated on top of the astrocyte bed. Astrocytes were stained for GFAP (blue), and neurons are stained for beta-III-tubulin (red). −200× total magnification, scale bar=100 microns.

FIG. 2 illustrates the successful disposition of multiple cell layers on a cylindrical surface of less than 250 μm, and depicts alignment of the cells along the long axis of the cylinder.

FIG. 3-

### DETAILED DESCRIPTION

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE I

Directional Outgrowth of Neurons on a Planar Substrate

With the following method, a device of the present invention may be made in essentially any shape that permits easy placement in the desired location in a subject or patient in which neurons would be desired such as in the damaged brain, spinal cord or a peripheral nerve. This example describes the construction of a planar device that contains the appropriate cell types for transplantation comprised of a suitable biomaterial that has a surface microtopography that is oriented with a specific directionality and is seeded with a first primary layer of primary astrocytes which then serves as a substrate for the attachment and alignment of a second layer of primary neurons. This example is not meant to be limiting in the scope of application or in the types of cells that may be utilized.

Production of the Oriented Substrates

Oriented surface finishes are prepared on appropriate sized electroformed solid nickel, titanium or other suitable machinable metal surface by one of several methods including but not limited to flat lapping, grinding, milling or turning to produce a surface finish with an average surface roughness of at least 4 microinches but not exceeded 64 microinches with a surface texture made in one direction to produce an oriented surface microtextures. The oriented surface topography is then transferred to any suitable biomaterial for example polypropylene by a thermomolding procedure. A smooth film of polypropylene formed by melt extrusion is clamped to the metal surface finish and placed in a chamber at 200 degrees C. for 3-5 minutes or until the materials appear to have uniformly melted on the surface. The piece is then removed, allowed to cool for a few seconds and then dipped into water at room temperature. The plastic piece is removed from the metal surface and transfer of the appropriate surface finish from the metal to the plastic part is achieved. This method may be used with any suitable thermoplastic biomaterial.

Astrocyte Preparation

Mixed cerebral cortex was isolated from postnatal day 1 rats. Briefly, meningeal tissue was removed under a dissection microscope and the cleaned cortices were placed in a droplet of L15 medium (Gibco) and mechanically dissociated using scalpels. Tissue was placed in L15 containing 0.35% collagenase (Sigma) for 30 minutes. After centrifugation at 600 g for 3 minutes, the tissue was resuspended in Hanks Balanced Salt Solution containing 0.08% trypsin/0.0125% EDTA (Sigma) for 30 minutes. Following digestion, SBTI-DNase (0.53 mg/mL soybean trypsin inhibitor, 0.04 mg/ml bovine pancreatic DNase and 3 mg/mL BSA fraction V; Sigma) was added in a 1:2 ratio to the digestion solution for an additional 5 minute incubation. The solution was centrifuged at 600 g for 5 minutes and resuspended in a small volume of DMEM-FBS (DMEM with 10% fetal bovine serum, 2 mM glutamine and 25 4 g/mL gentamycin) and 0.1% DNase (Worthington) and triturated through fire-polished pasteur pipettes followed by a 1 cc syringe with needles of decreasing diameter. The resulting cell suspension was centrifuged at 1000 g for 5 minutes, resuspended in DMEM-FBS and grown in DMEM-FBS. When near confluence, the flask cap was sealed tight and placed in a shaking incubator overnight set at 37° C. and a shaking speed of 175 r.p.m. Following the shake-off, cells were treated for two days with 20 uM cytosine arabanoside (Ara-C; Sigma). The resulting cell population is composed almost entirely of type-1-astrocytes. The astrocytes were removed from the flask and seeded on the oriented sterilized substrates prepared as described above. The astrocytes were allowed to grow to a monolayer and then seeded with primary dorsal root ganglion neurons.

M. Noble and K. Murray, “Purified Astrocytes promote the In Vitro Division of A Bipotential Glial Progenitor Cell,” EMBO J., 3:2243-2247 (1984).

Preparation of Dorsal Root Ganglion Neurons

Dorsal root ganglion neurons (DRG) were prepared from postnatal day 1 rats. Briefly, dorsal roots were individually removed from the spinal column and placed in a dish of ice-cold L15 medium (Gibco BRL). The nerve roots were stripped from the bodies, and the remaining ganglia bodies were placed into a solution of L15 containing 0.33% w/v collagenase (Sigma) for 45 minutes. The collagenase-digested suspension was centrifuged at 600 g for 3 minutes then placed into a solution of DMEM containing 0.25% w/v and 0.1% DNase for 30 minutes. The digested tissue was again centrifuged at 600 g for 3 minutes then resuspended in a small volume of DMEM containing 0.1% w/v DNase. The suspension was triturated with fire-polished pasteur pipettes of decreasing bore diameter, centrifuged at 1000 g for 5 minutes and resuspended in 1 mL of DMEM containing antibody against the ganglioside 04 (1:100) and 10% rabbit complement (Sigma) for 30 minutes. This is a purification step to remove contaminating Schwann cells from the suspension. The suspension was diluted to 10 mL with DMEM and centrifuged at 1000 g for 5 minutes followed by 3000 g for 1 minute. Cells were resuspended in DMEM-F12 supplemented with SATO and 10 ng/mL 2.5S nerve growth factor (NGF, Gibco BRL) and 50 uM diI(C18) (Molecular Probes) for 15 minutes to fluorescently mark the neurons prior to plating on astrocytes. The device is completed by allowing the neuronal cells to extend their axons over an appropriate time scale for the desired application. Again, these types of neurons and other cell types are illustrative and are not meant to be limiting. The device is then surgically implanted into the damaged portion of the nervous system to effect repair by any means as those skilled in the art would choose.

### EXAMPLE 2

Directional Outgrowth of Neurons on A Filamentous Substrate

With the following method, a device of the present invention may be made in a cylindrical or filamentous geometry of essentially any length that permits easy placement in the desired location of a subject or patient in which of neurons would be desired such as in the damaged brain, spinal cord or peripheral nerve. Preferably several filaments would be bundled together in a semipermeable hollow fiber. This example describes the construction of such a device that contains the appropriate cell types for transplantation which is comprised of any suitable biomaterial filament of diameter of less than 200 microns that is seeded with a first or primary layer of primary astrocytes or other appropriate cell type that supports the attachment of a second layer of primary neurons that are aligned in the direction of the long axis of the cylindrical substrate. This example is not meant to be limited in scope of application or in the types of cells that may be utilized.

Production of the Cylindrical or Filamentous Substrates

ilaments are fabricated by pulling molten polypropylene from a melt extruder at different take-up speeds. A subset of these materials are then used to fabricate filaments with oriented surface microtopography by a treatment that involved straining (change in length) the fibers 350% at a velocity of 0.1 in/s. The straining treatment causes the fiber to neck to a smaller diameter and induces the formation of surface microtopography or microtexture that is generally aligned with the long axis of the fiber.

The fibers are cut and fixed onto a small stainless steel frame using a biocompatible UV adhesive. The fibers are cleaned to remove debris and oils by washing in 1% alconox and chemically sterilized by soaking in 70% ethanol for 1 hour. A poly-1-lysine (PLL) coating was applied by incubating the fibers in a 50 μg/ml solution of PLL for at least 1 hour. Subsequently, a laminin coating was added again by incubating the filaments for at least 1 hour (laminin, 20 μg/ml) in PBS. The filaments were seeded with astrocytes using the methods described above. Following an appropriate period, the cells to attach and grow a secondary layer of neurons is added to the construct. Following an appropriate growth period, the cell covered filaments are packaged into the lumen of a semperable hollow fiber of the type used in the cell encapsulation field having a MWCO of 100-2000 KD and being composed of a biocompatible material such as polyacrylonitrile-polyvinyl chloride or polysulphone or other suitable material. The construct is then ready for placement into damaged brain, spinal cord or peripheral nerve by suture, fibrin glue or other suitable means as those killed in the art would choose.

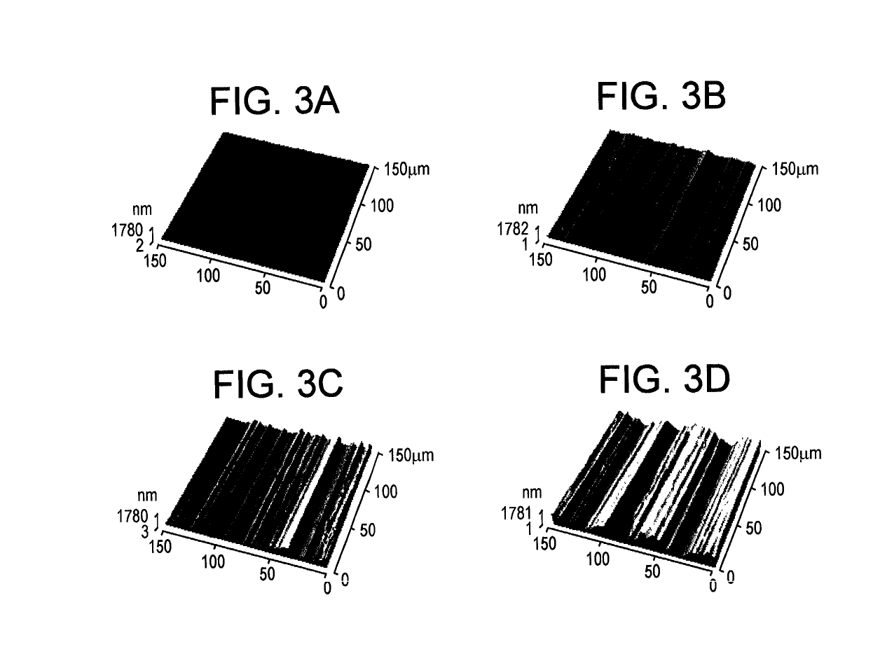

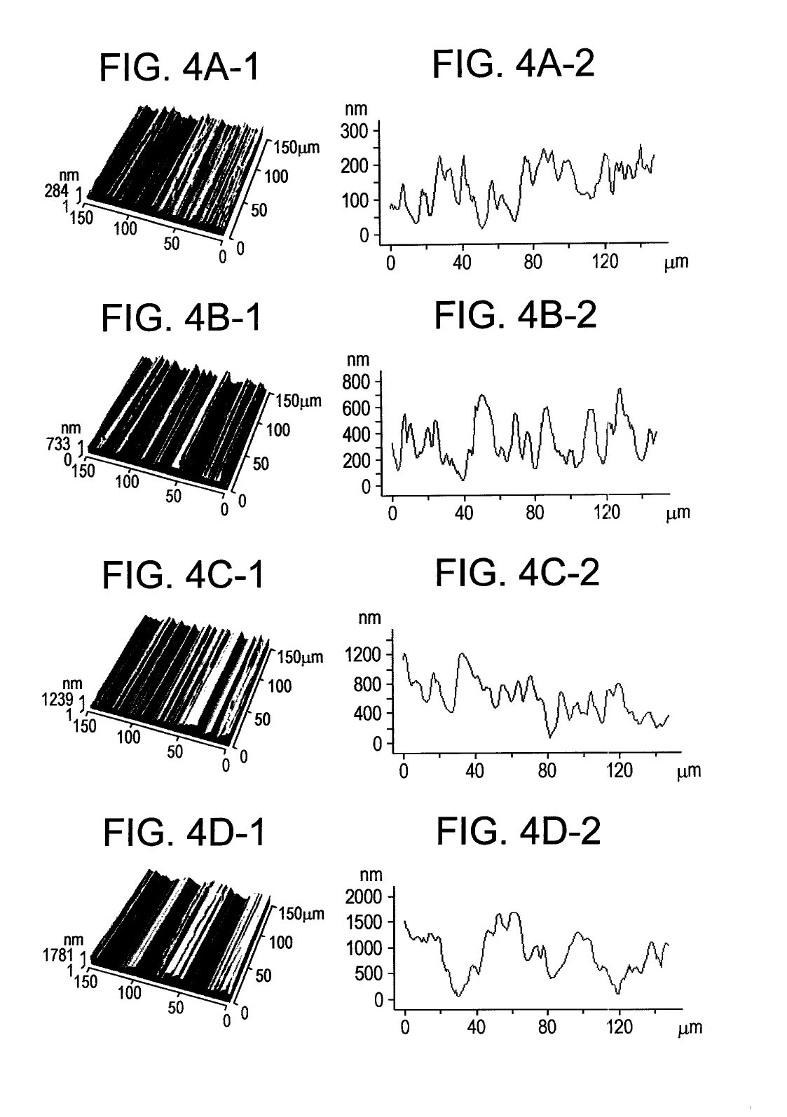

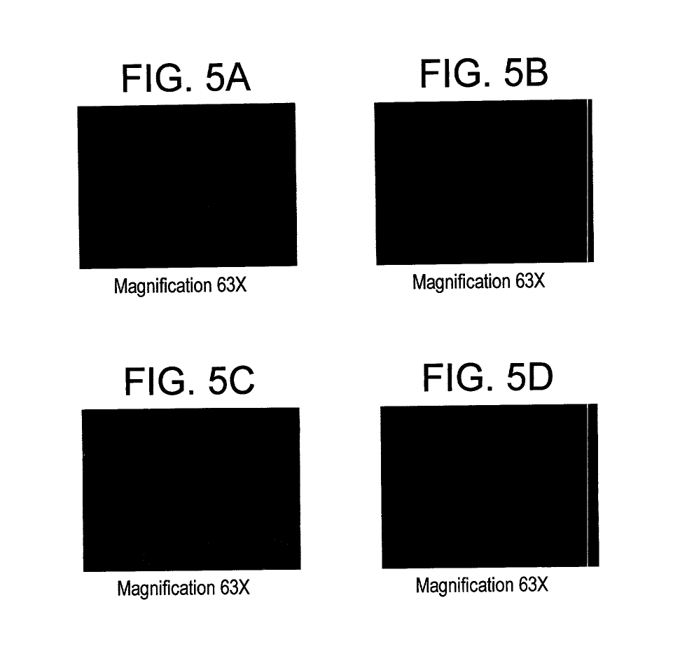

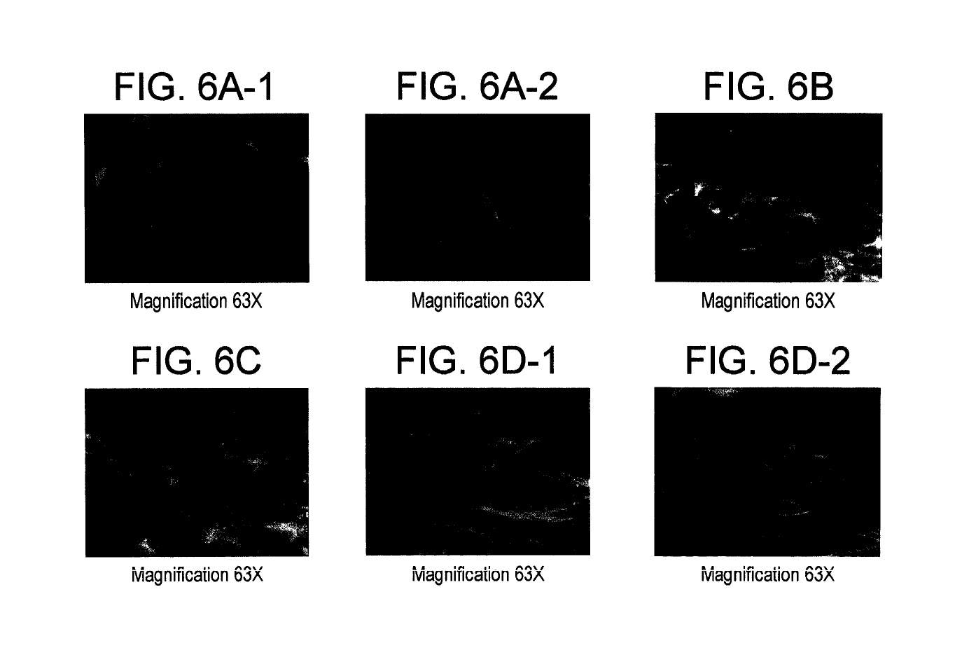

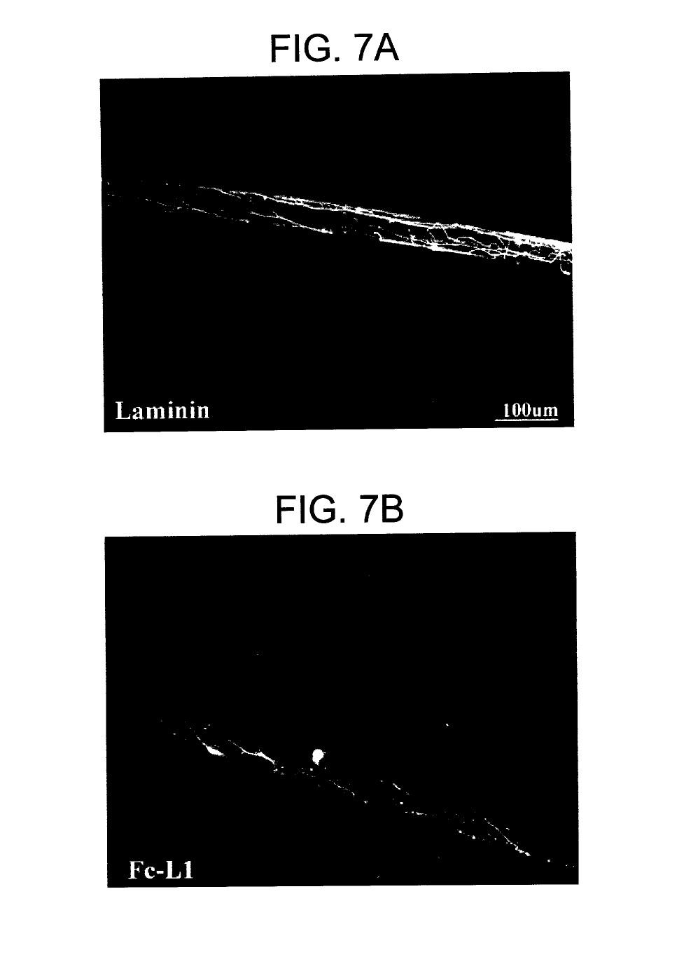

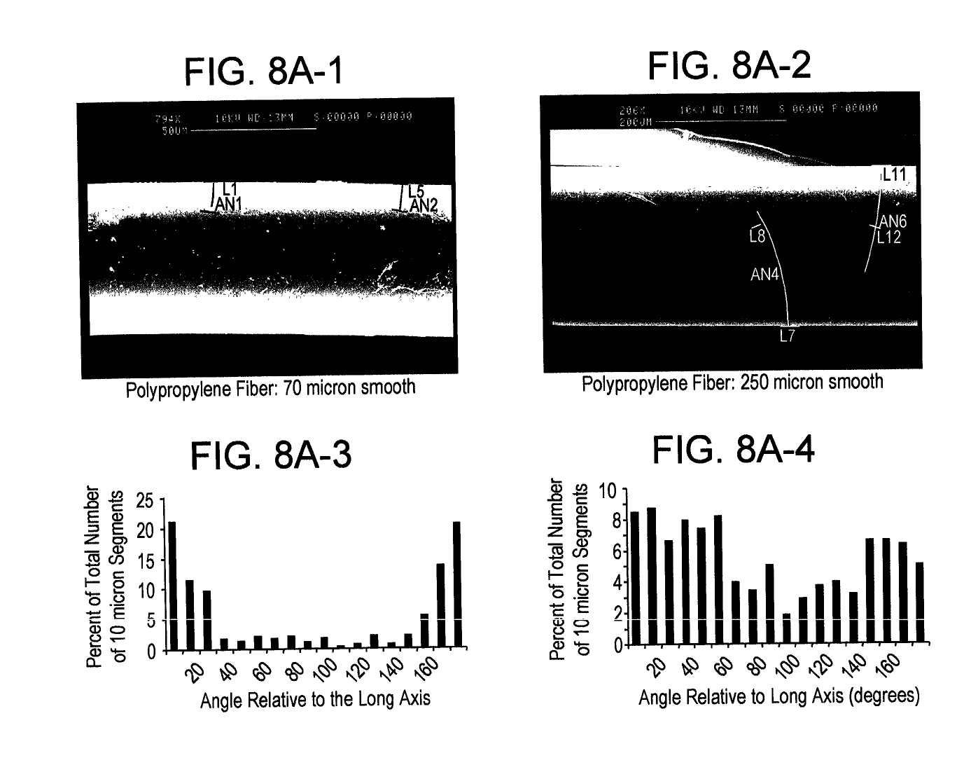

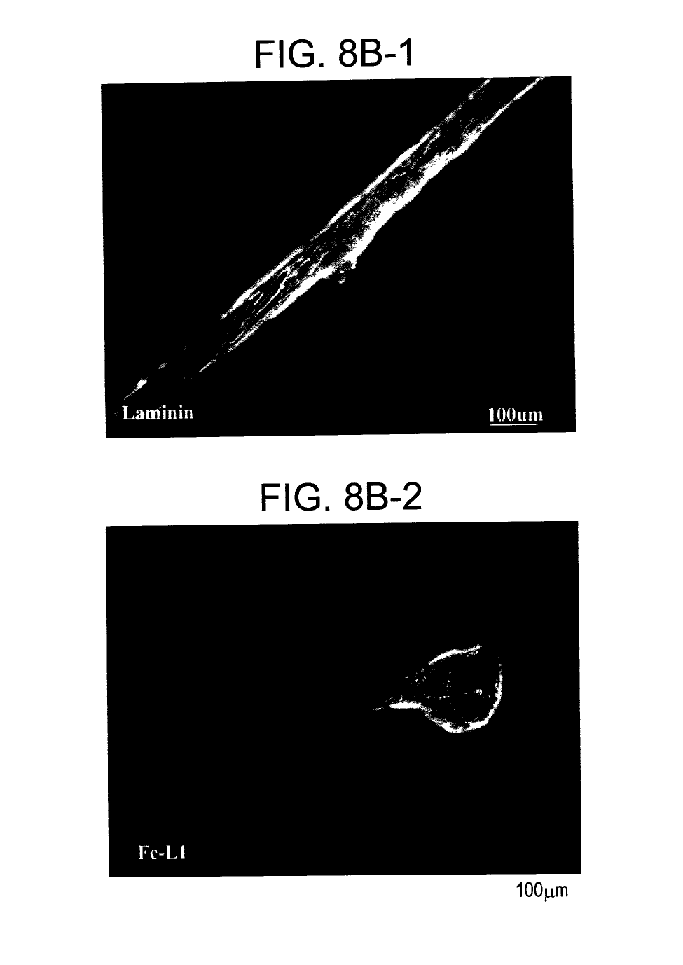

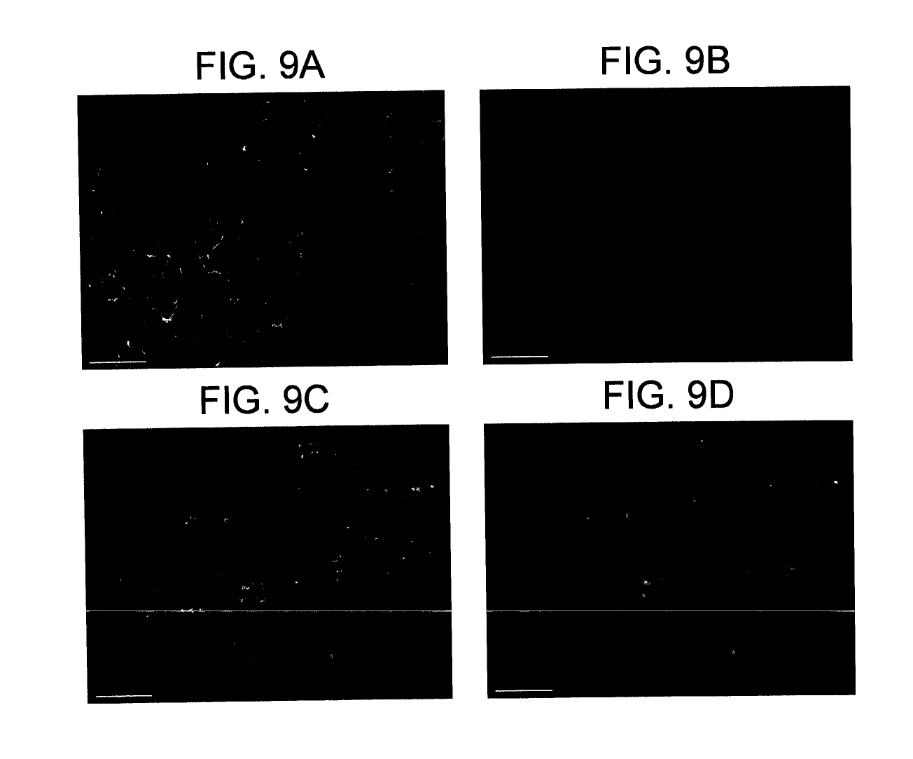

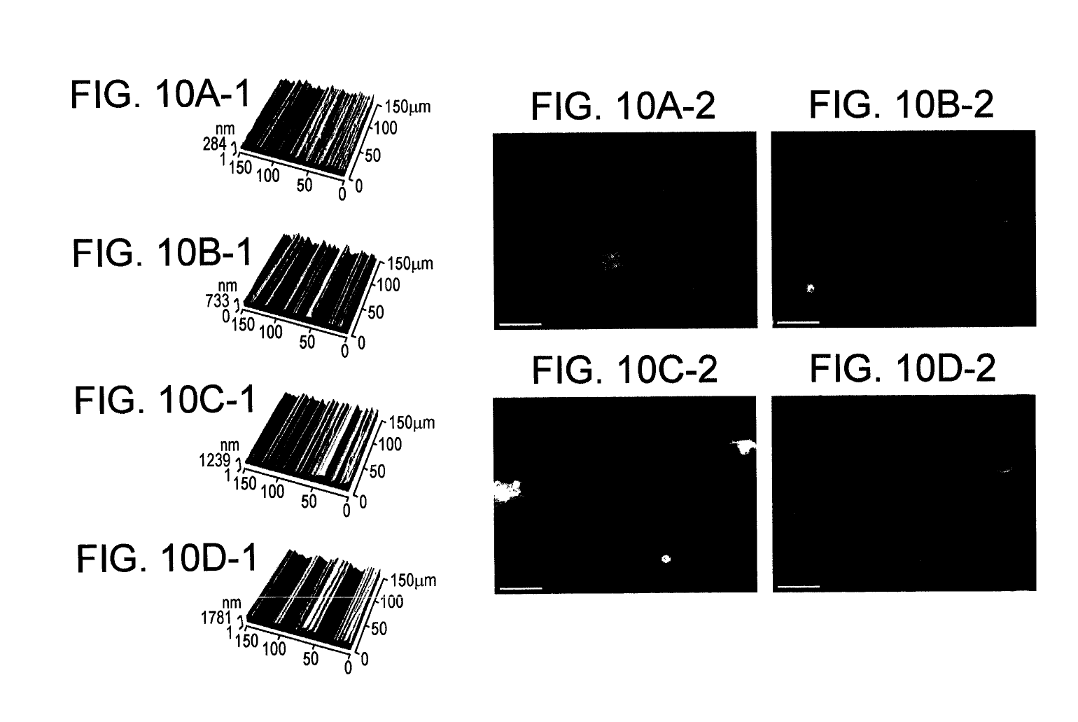

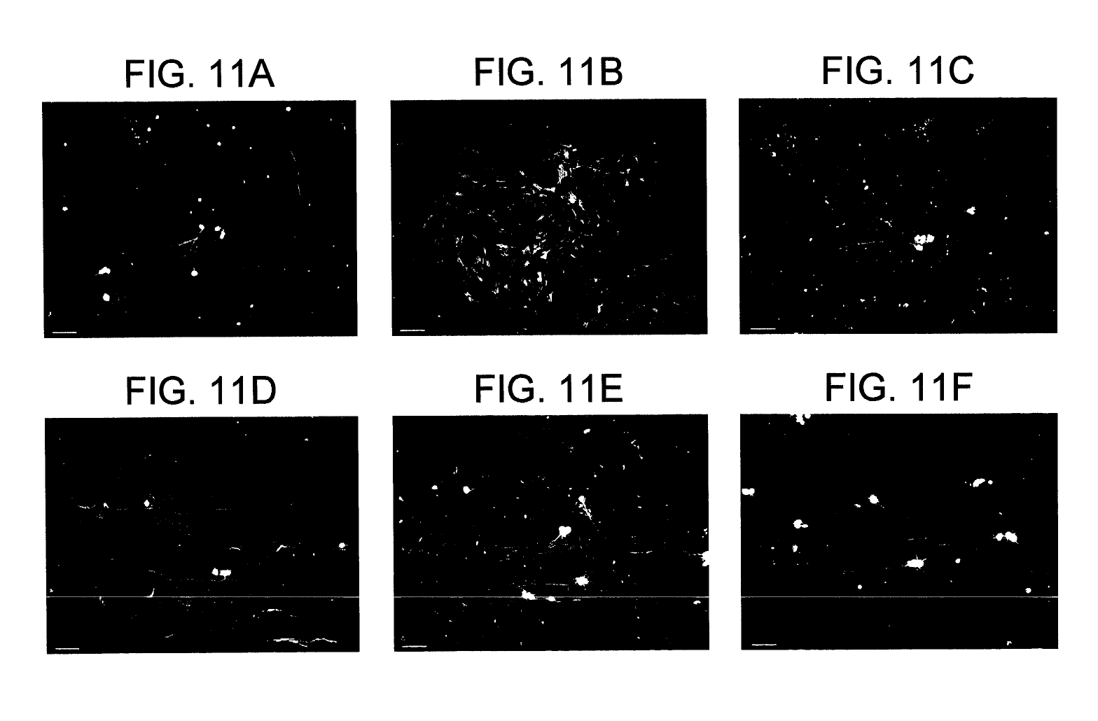

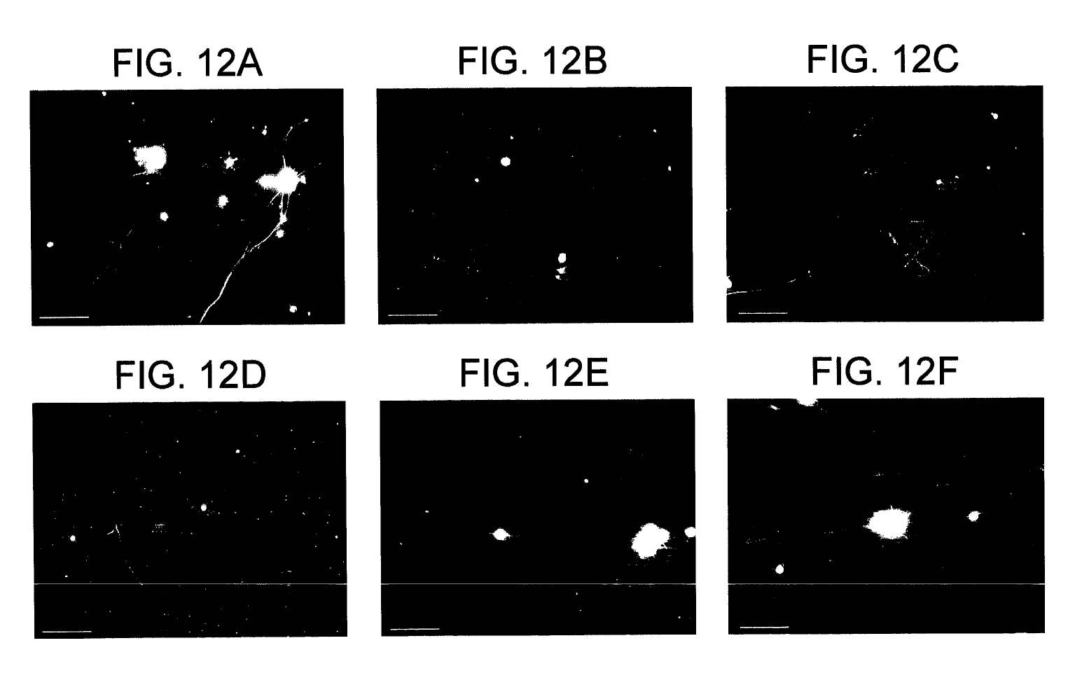

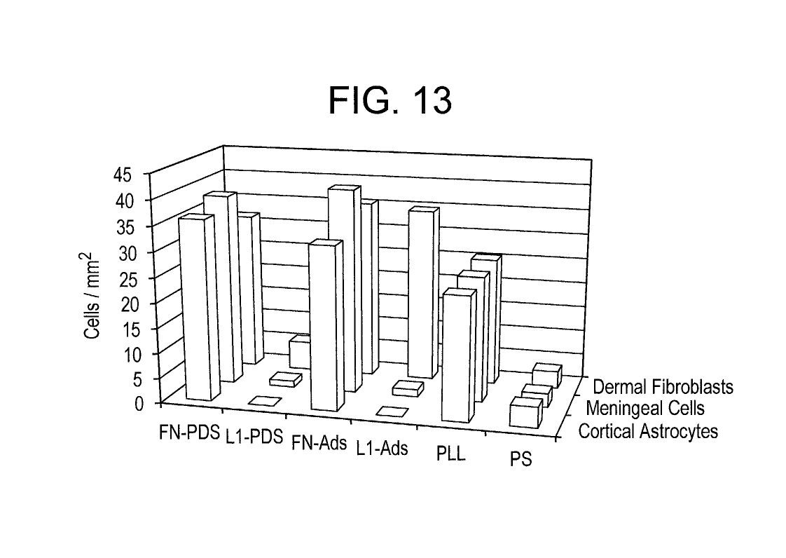

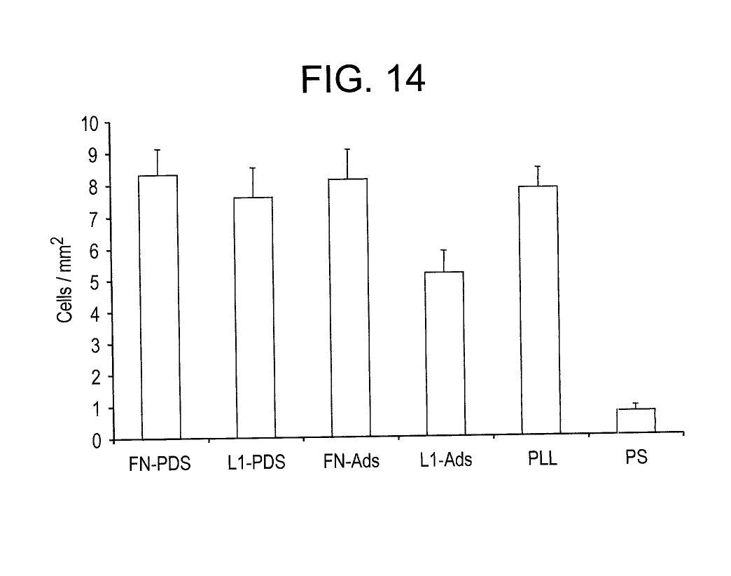

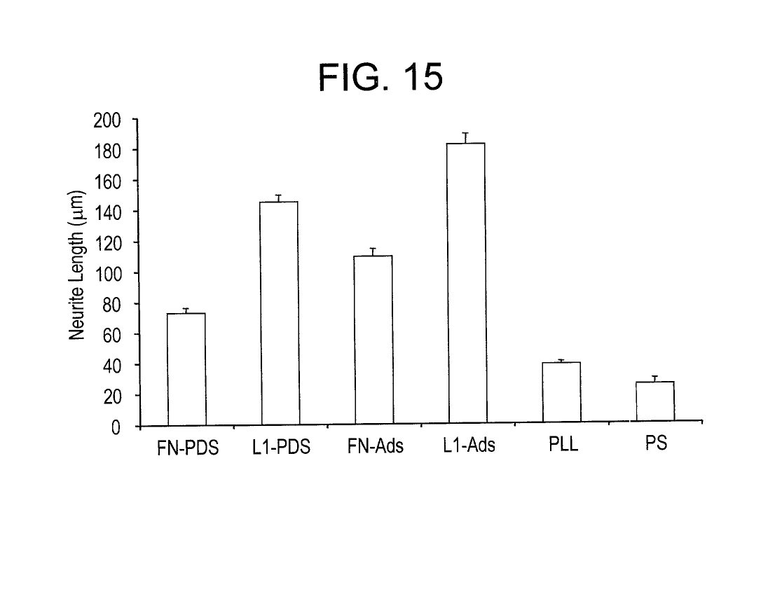

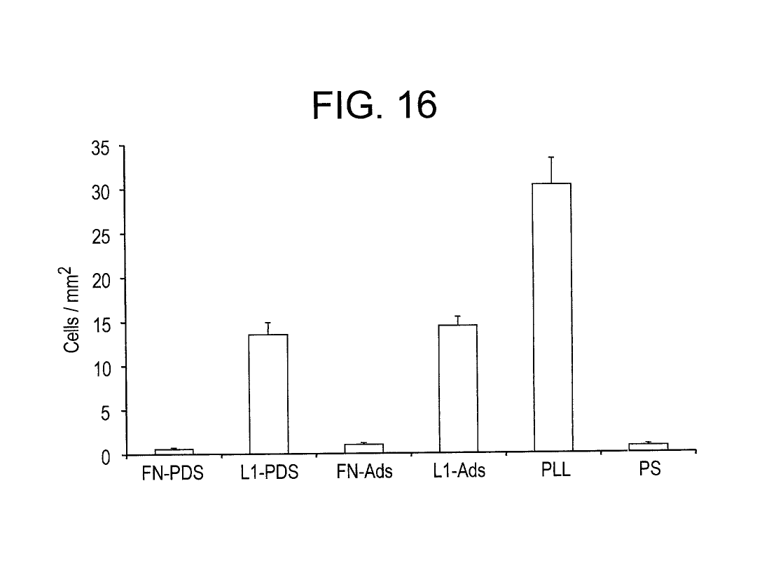

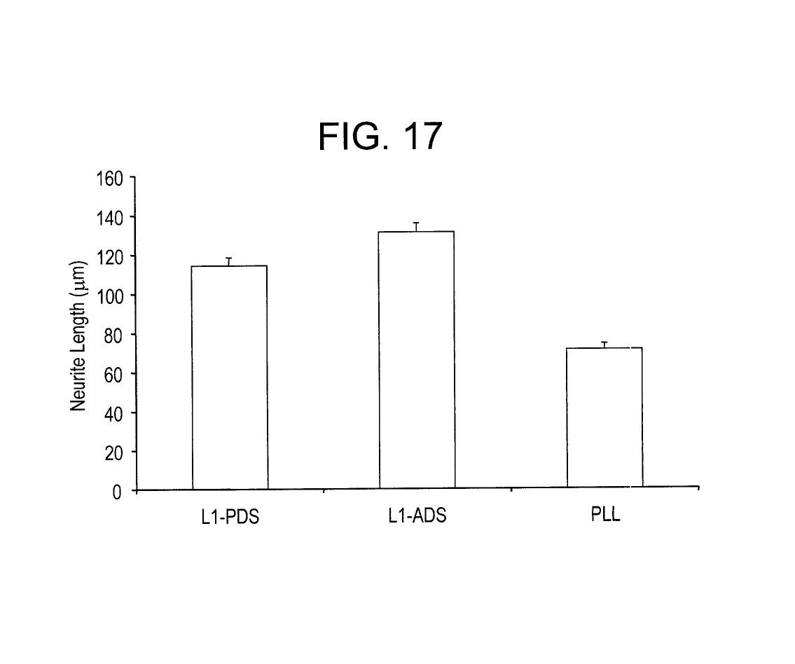

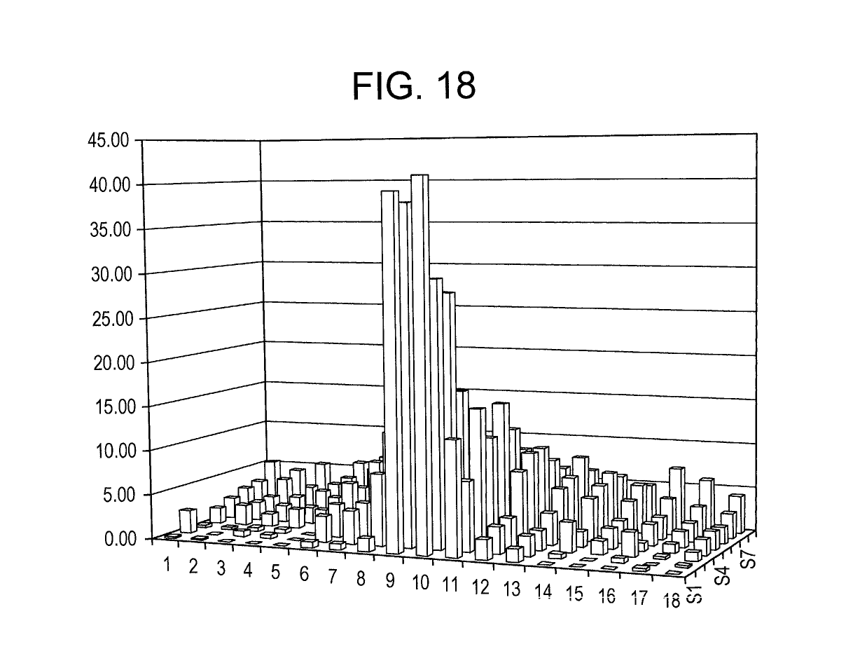

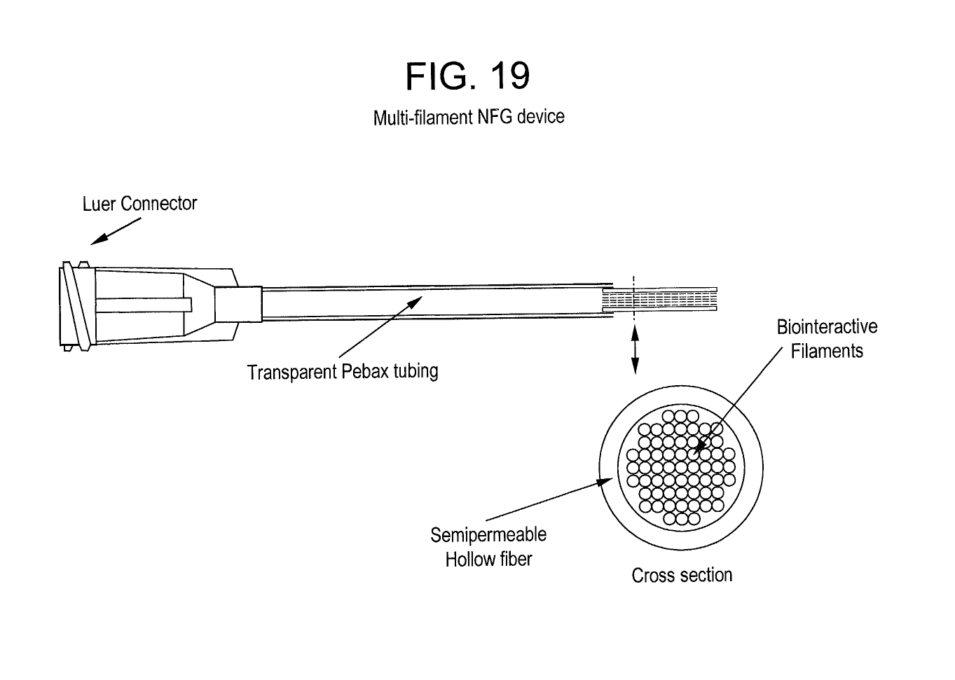

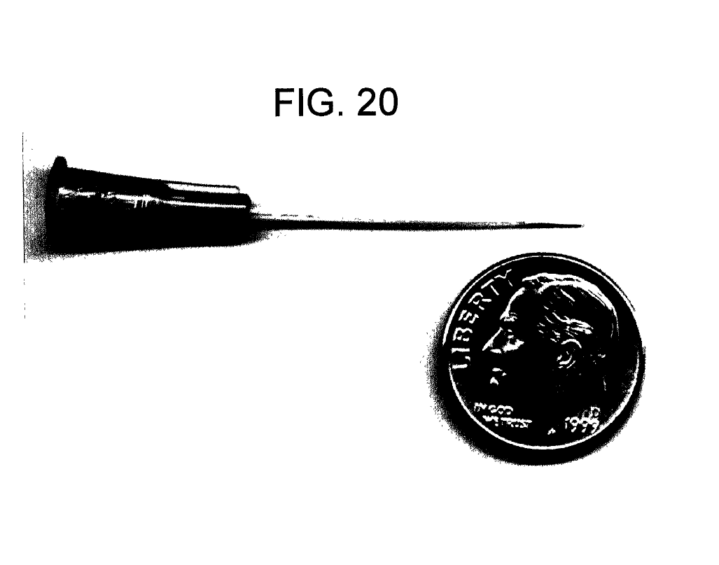

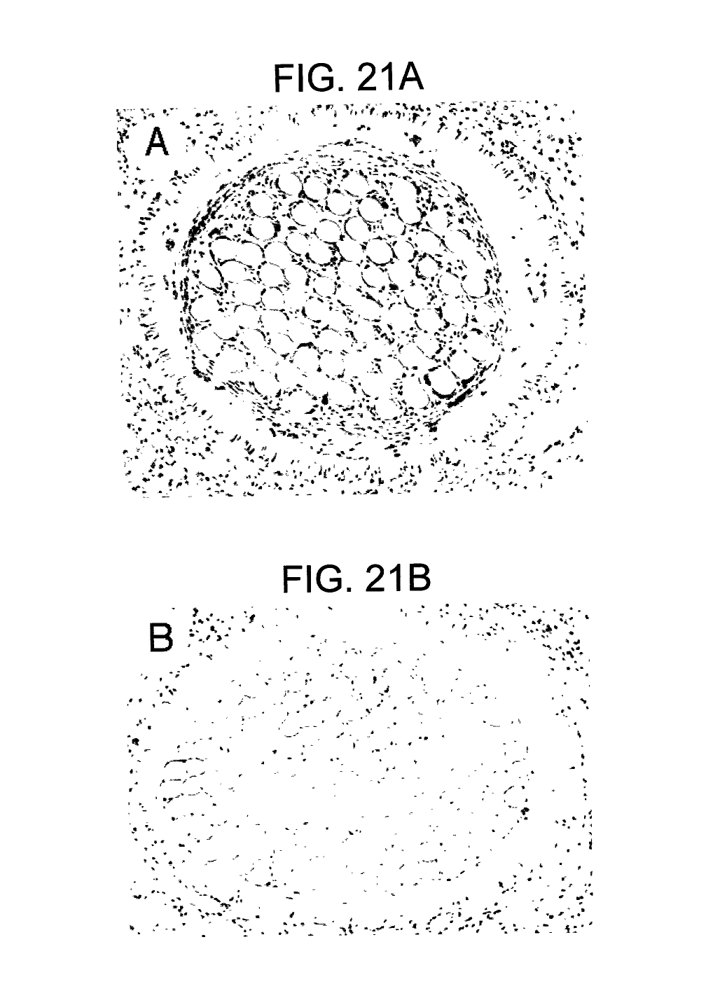

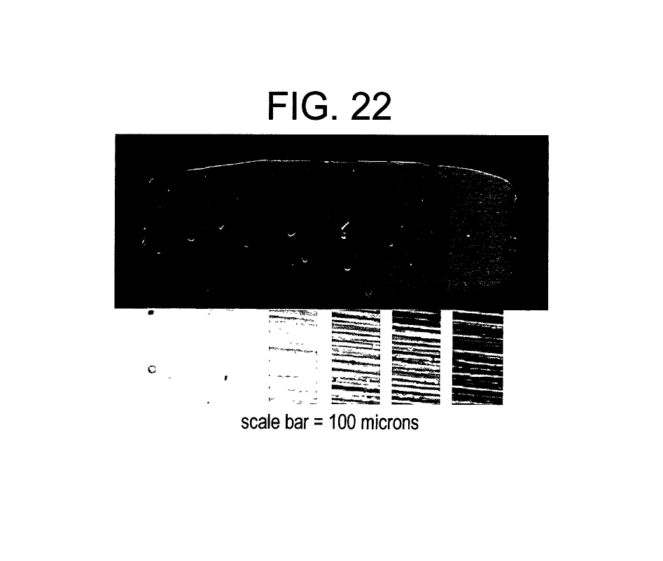

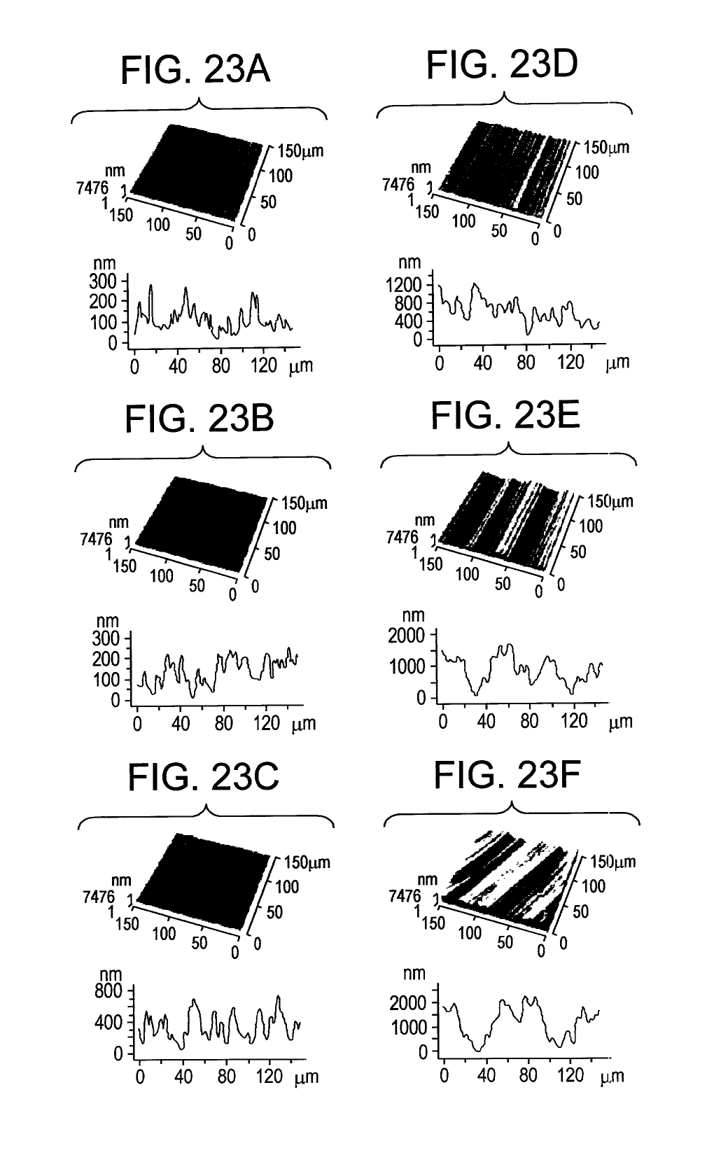

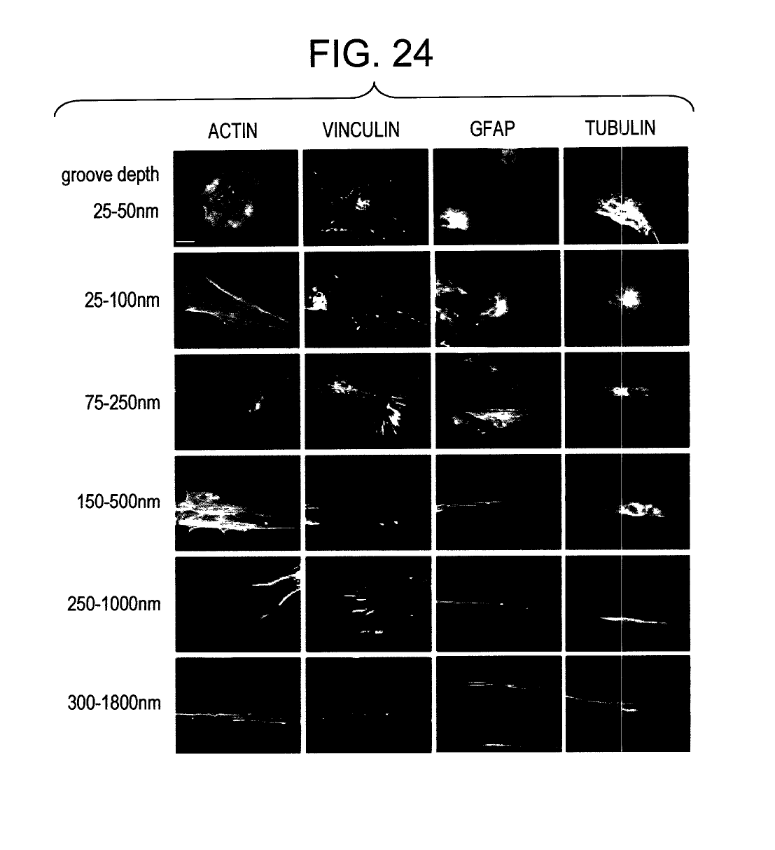

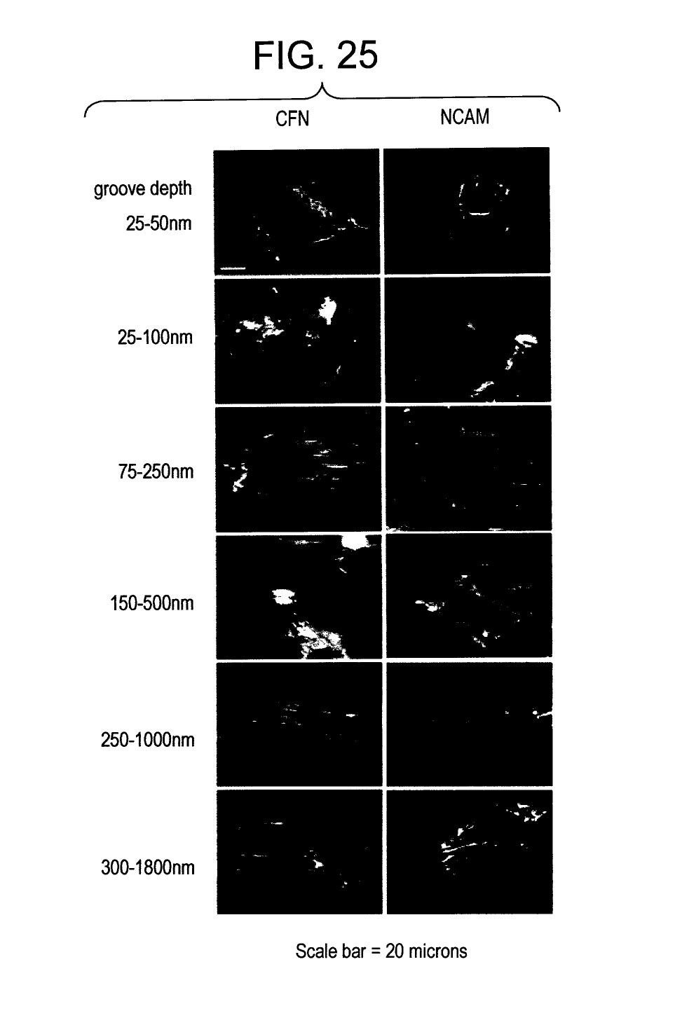

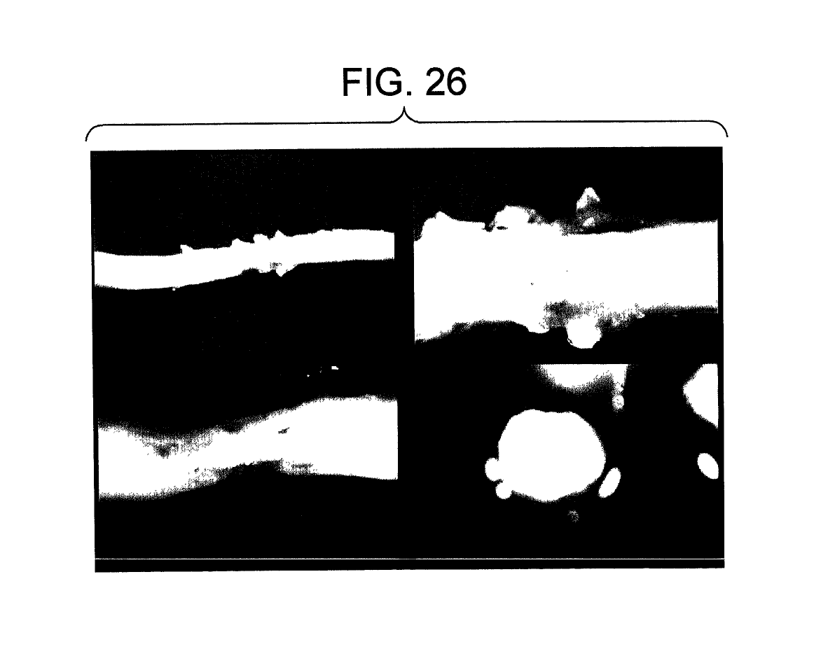

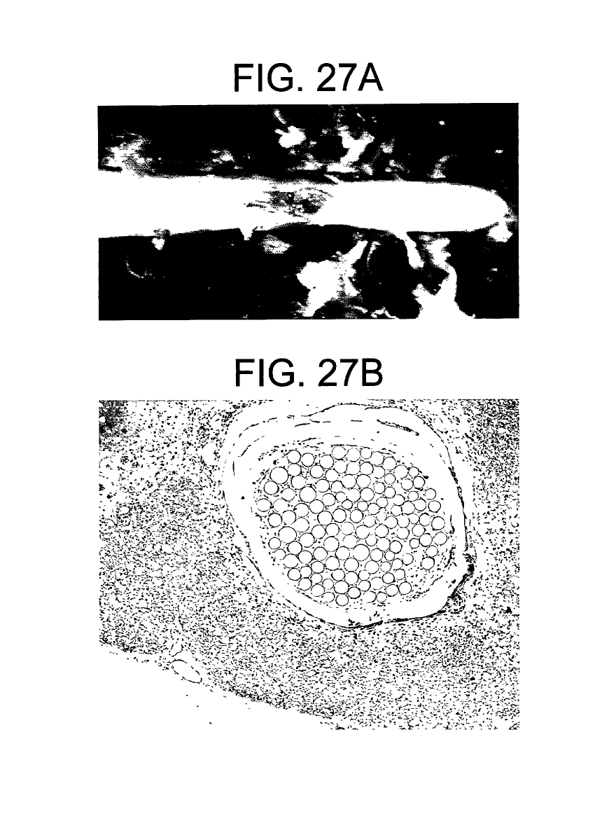

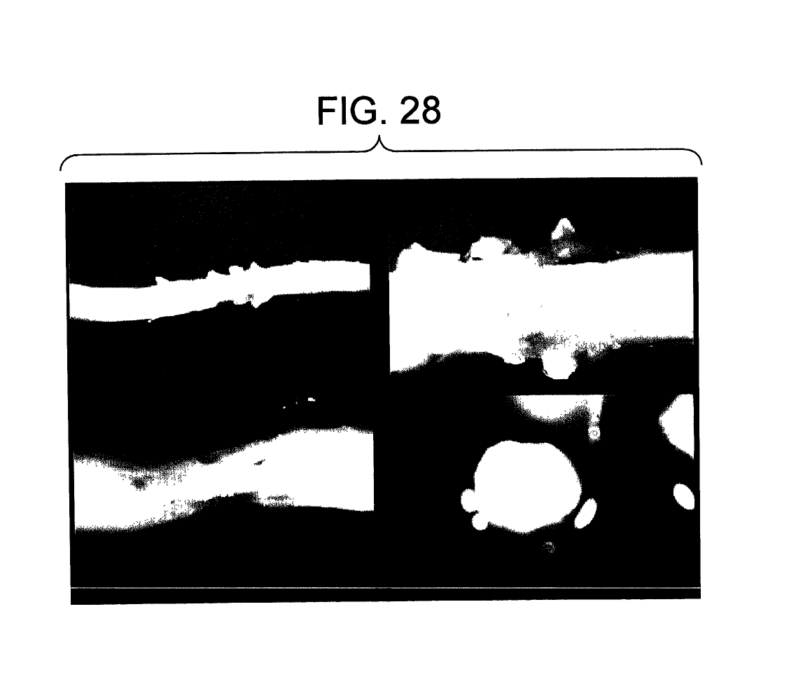

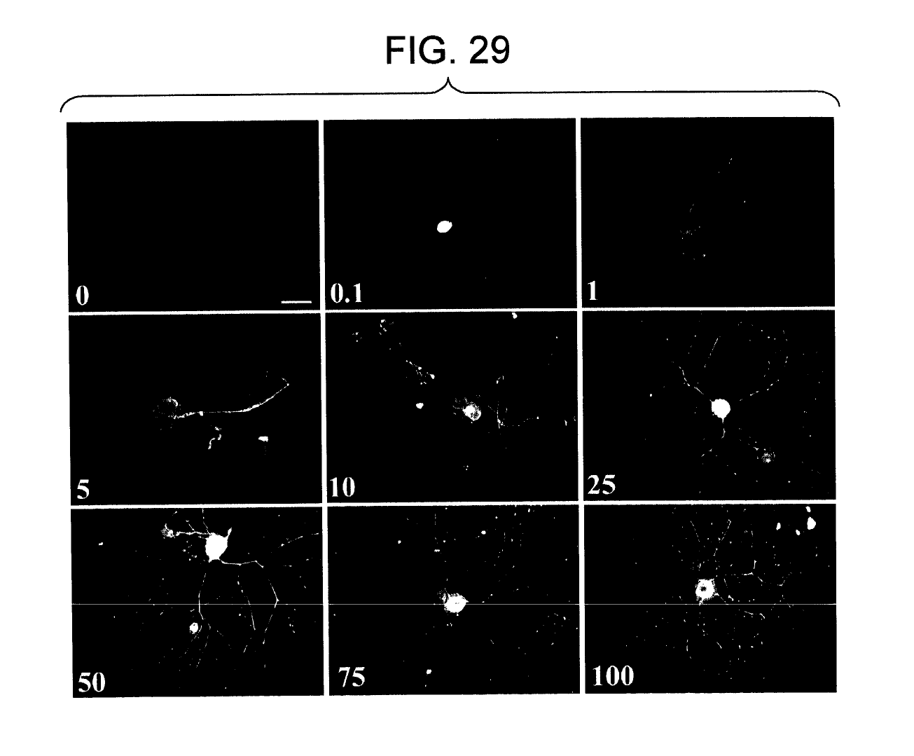

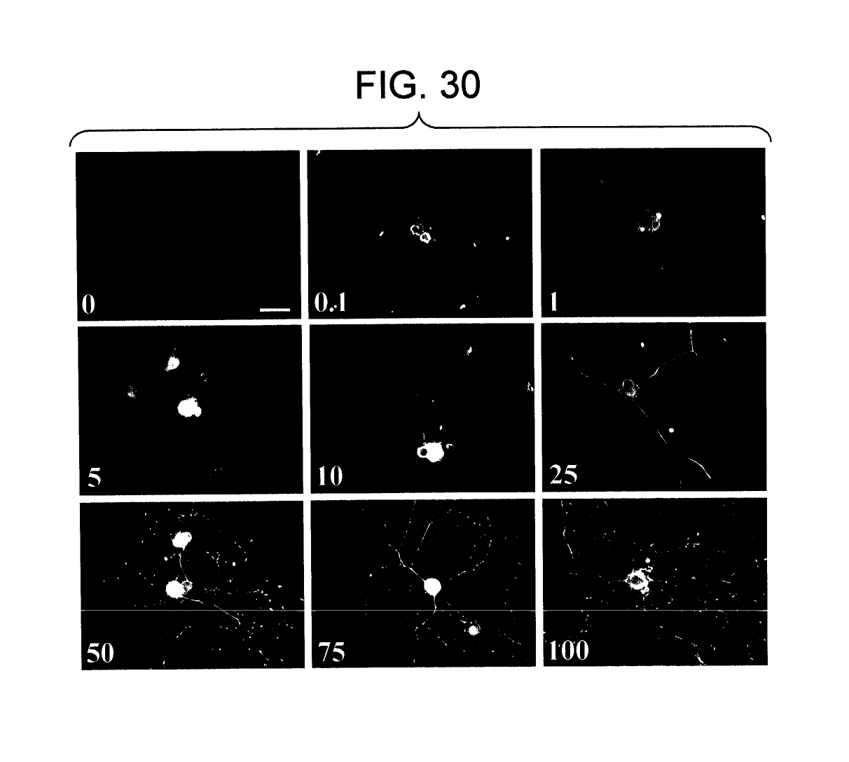

We have developed a novel method for creating microtexture on filaments of diameters down to 37 micron. Our results indicate that significant increases in neurite outgrowth can be achieved by growing primary neurons on materials with a fine surface microtexture in the range of 1.5 microns compared to materials with smooth surfaces in the same solution microenvironment whether on solid films (see FIG. 3) or on microtextured filaments (FIG. 4). This behavior appears to be preserved over a wide range of materials with differing surface chemistry treated with either the ECM proteins FN and laminin (LN) or the neural cell adhesion molecule (L1).

### EXAMPLE 3

In this study, we developed a simple method for creating NFG substrates with differing surface microtopographies. Our studies indicated that the method can be used with a variety of polymeric biomaterials, can be used to produce a series of oriented microtopographies on one surface in which all of the topographies run in a particular direction, and can be used to guide the development of. attached primary neurons. Scanning atomic force microscopy revealed that the method can be used to vary the surface roughness over an order of magnitude (FIG. 3). Thus far we have used the method to vary the surface microtopography from approximately a 2 micron surface roughness to a 200 nanometer smooth surface finish (FIG. 4). The results of cell behavioral studies indicated that cell orientation in the direction of the surface texture is optimal in the range or 2000-1200 nm. Smoother surface finishes do not elicit the same morphological orientation. Cells appear to be oriented randomly on the smoother surface finishes (FIGS. 5 & 6; results shown for meningeal cells only). Initial experiments indicate the similar behavior appears to hold for astrocytes and primary DRG neurons. Studies in progress are evaluating the usefulness of the approach in enhancing directed neurite outgrowth in the injured spinal cord and the possibility of creating gradients of surface microtopography to direct cell behavior.

### EXAMPLE 4

L1-Materials Studies:

In this study, we compared the behavior of primary neurons and astrocytes seeded onto the surface of NFG filaments that possessed a oriented surface microtopography and were surface coated with either the extracellular matrix protein, laminin or the cell adhesion molecule, L1. Our studies revealed that primary DRG neurons extend βb-III tubulin positive neurites equally well on both materials (FIG. 7). Interestingly, however, primary astrocytes did not prefer the L1 coated filaments. Whereas astrocytes readily attached to form a confluent monolayer on laminin coated filaments as observed by the staining pattern for the intermediate filament GFAP, they only attached minimally to the L1 treated filaments (FIG. 8). On the L1 treated filaments, astrocytes appeared to prefer to attach to one another and were most often observed in aggregated clumps or clusters. Similar experiments are in progress with other relevant cell types to examine whether this behavior generalizes to meningeal cells and fibroblasts. The specificity of L1 may provide a method for regulating the behavior of regenerating neurons on the NFG implants.

### EXAMPLE 5

Cell Behavior on Controlled Microtopographies:

Thus far our cell culture studies on oriented microtopographies have focused on characterizing the behavior of several primary cell types including neurons, astrocytes and meningeal cells on flat substrates and filaments of varying surface topography. As mentioned in earlier reports, these cell types are being studied because of the important role that they play in both the wound healing and the nerve regeneration response of the injured spinal cord.

The results indicate that each of the various cell types responds to oriented surface microtopography by changing cell morphological features so as to align with the direction of the underlying surface grooves. Moreover, the change in overall cell morphology is accompanied by several intracellular changes such as rearrangement of the cytoskeleton. Although the organization of actin microfilaments or stress fibers and focal adhesions appear to be the most strongly affected, intermediate filaments and microtubule directionality are also influenced, so that they align with the direction of the underlying surface grooves. Our studies using surfaces with varying groove depths have specified the limits of this surface-induced cell orientation at approximately 400 nm, below which cells appear to be much less sensitive to the orientation of the underlying surface grooves.

Effect of Varying Diameter and Surface Roughness of Filaments on the Directionality of DRG Outgrowth

The results clearly indicate that surface texture is an important determinant of adherent cell behavior for biomaterials with relatively flat solid surfaces. However, in cases where the biomaterial takes the shape of a filament or a larger fiber, the parameters that influence cell behavior are much less clear. In these cases, at least two variables are potentially at play, namely, surface texture and the radius of curvature of the material. To determine the relative importance that that each of these respective factors play, we initiated a series of studies that sought to examine the relative contribution of each factor on the behavior of adherent cells.

Using filament extrusion and the mechanical testing system described in earlier progress reports, we fabricated a series of polypropylene (PP) filaments of varying diameters and surface microtextures. Filaments with smooth surfaces were fabricated by pulling molten polypropylene from the extruder at different take-up speeds. A subset of these materials were then used to fabricate filaments with oriented surface microtopography by a treatment that involved straining (change in length) the fibers 350% at a velocity of 0.1 in/s. The straining behavior was chosen because it correlated with the original procedure used to fabricate the filament used for our in vivo analyses. The straining treatment causes the fiber to neck to a smaller diameter as well as induces the formation of surface microtopography or microtexture that is generally aligned with the long axis of the fiber

A range of filament diameters was selected for experiments, from 42 μm to 680 μm, with filaments being paired into groups with similar diameter but with either smooth or textured surfaces. For our studies, the fibers are cut and fixed onto a small stainless steel frames using a biocompatible UV adhesive. The fibers are cleaned to remove debris and oils by washing in 1% alconox and chemically sterilized by soaking in 70% ethanol for 1 hr. A poly-1-lysine (PLL) coating was applied by incubating the fibers in a 50 μg/ml solution of PLL for at least 1 hr. Subsequently, a laminin coating was added again by incubating the filaments for at least 1 hr (laminin, 20 μg/ml) in PBS.

Cell material interaction studies were first initiated with primary DRG neurons.

Studies with other relevant cell types will follow. The DRG's were plated onto the filaments at a density ranging from 50,000-75,000 cells/ml. Two ml of cell suspension was used to cover the frames in a 12 well culture plate, which was non-adhesive for tissue. The cells were allowed to grow for 36 hrs in an incubator. The cells were then fixed by methanol treatment. The cells were stained using an antibody to neurofilament and visualized by a secondary antibody to Texas Red.

The cell seeded biomaterials were then prepared for analysis by scanning electron microscopy (SEM) by an osmication and a dehydration procedure. SEM photographs were taken of all filament surfaces with attached DRG's. The images were imported into an image processing program. To quantify the directionality of the neurites, each extension from the cell body was broken into 10 μm lengths. The angle of each segment was measured relative to the direction of the long axis of the fiber (the edge of the fiber taken from the SEM image was used as an indicator of the angle to the long axis). A histogram of segment angles is generated using a bin size of 10 degrees and a range of 0 to 180 degrees. Histograms are generated for each fiber size in both the strained and unstrained category.

The results of the initial studies indicate that, in general, neurite directionality is influenced by both the radius of curvature as well as by the surface texture of the filament. For filaments below 200 micron diameters the direction of DRG neurite outgrowth appears to be more strongly influenced by the declining radius of curvature than by the surface texture as smooth surfaced filaments of this dimensional range induce directional outgrowth along the long axis. In general, filaments with diameters above 250 micron diameters display more random patterns of neurite outgrowth. Surface texture appears to be a more important factor for promoting directed neurite outgrowth on filaments with diameters greater than 250 microns. We have included a figure as an example of the type of data these studies are generating (FIG. 8). Most of our efforts during the quarter were focused on fabricating the different biomaterials and developing the test protocol for analysis of directed neurite outgrowth. These studies are ongoing and will be disclosed as they are completed in subsequent reports.

### EXAMPLE 6

Directional Cell Growth of Multiple Cell Layers on Biomaterials With an Oriented and Nonuniform Surface Microtopography:

As mentioned above, most of our studies of cell-materials interations have been focused on using monolayers of several cell types employing standard 2-D cell culture technique. In one of our studies examining the behavior of primary meningeal cell behavior on oriented microtopography of varying groove depth, we discovered that the pericellular fibronectin matrix, that is, the fibrillar matrix secreted by the meningeal cells and organized on their upper surface of the cell monolayer was also aligned in the direction of the underlying surface grooves, suggesting that the topographic information was somehow transduced through the overlying cell monolayer to the secreted matrix.

Since primary DRG neurons bind to pericelluar fibronectin we asked whether the underlying topographic cue could be due to a second cell layer, namely the DRG's seeded on top of a confluent layer of meningeal cells. The results indicated that it is indeed possible to confer directional information from a biomaterial surface through one cell layer to direct the behavior of another cell layer seeded on top of the first (FIG. 9).

Since primary meningeal cells plays a major role in the scarring reaction of spinal cord injury, we initiated another series of studies in which we asked whether monolayers of primary astrocytes seeded onto the surface of biomaterials with oriented surface grooves could also produce the directed outgrowth of DRG's seeded on their upper surface. The data indicated that not only was the information transferred through the astrocyte monolayer but that the sensitivity to groove depth was transferred as well, suggesting that textured biomaterials may be used to impart directionality to multiple layers away from the surface (FIGS. 10, 11, 12). This discovery has the potential to turn into technology with commercial applicability in both the research as well as the clinical sector, and is being treated accordingly. Ongoing studies in this area are attempting to uncover the mechanistic details underlying this phenomena and will examine the utility of the biohybrid approach in our various transplantation paradigms, using relevant cell types.

### EXAMPLE 7

Cell Behavior on Biomaterials With Controlled Surface Ligand Density and Directional Microtopographies:

We have continued our investigations of cell material interactions using material surfaces modified with biological signaling molecules such as fibronectin, laminin, and the cell adhesion molecule L1. Our efforts have been split between studies that examined the biological specificity of various ligands, and studies that examine how the radius of curvature of filaments affects the directional outgrowth of neurons and other accessory cell types. The studies, described briefly below, strongly suggest that combining protein immobilization with controlled surface microtopography and geometry can be used to engineer novel biomaterials that not only permit certain cell behaviors, but also are capable of instructing certain types of cells to adopt specific and reproducible behaviors. The term “biointeractive materials” has been suggested to describe this new class of tissue engineering materials.

I. Neuron-Selective Growth on Biomaterials: Immobilization of L1

During the past quarter, significant effort has been focused on optimizing the immobilization of human L1 to biomaterial surfaces, and examining whether immobilization of L1 to a polymeric substrate would support neuronal cell attachment and neurite outgrowth in a cell-type selective manner in a serum containing environment. The results were compared to other substrate treatments including the immobilization of extracellular matrix molecules and coating with poly-1-lysine, which have been shown to be promiscuous binding ligands for multiple cell types.

For these studies, proteins were immobilized by physical adsorption to the biomaterial surface or by covalent immobilization. Covalent immobilization was accomplished through an activated surfactant coating method described in several of our previous progress reports. Briefly, pluronicä F108 (BASF) was modified to express terminal reactive pyridyl disulfide (PDS) groups and adsorbed to polypropylene (PP). A recombinant fusion protein of human L1 with an Fc immunoglobulin domain was used. Prior to immobilization, L1-Fc (400 μl, 2.47 mg/ml) was reduced by addition of 10 μl of 25 mM dithiothrietol (DTT) for 1 hour. The protein was separated from excess DTT on a PD-10 column (Pharmacia) equilibrated with 0.1 M phosphate buffer, pH 6.0. Bovine fibronectin (Sigma) was thiolated and served as a control. 96-well unmodified polystyrene plates (Nunc) with PP inserts were sterilized with 70% ethanol for ½ hour, then coated for 18 hours with 1% (w/v) F108-PDS. After rinsing with sterile distilled water, the plates were coated for 18 hours with 100-150 μg/ml thiolated fibronectin or reduced L1-Fc in 0.1 M phosphate buffer, pH 6.0. Thiolated fibronectin, reduced L1-Fc, and poly-D-lysine (PLL, 0.5 mg/ml) were adsorbed to untreated polystyrene wells to serve as controls. All wells were rinsed three times with Dulbecco's phosphate buffered saline prior to seeding.

Primary astrocytes, meningeal cells, dermal fibroblasts, cerebellar granule neurons (CGN) and dorsal root ganglion neurons (DRG) were obtained from postnatal rats. Astrocytes, meningeal cells, and fibroblasts were seeded at 1500 cells/well in DMEM-F12 (Gibco) with 10% fetal bovine serum (FBS) or SATO chemically defined media and 25 μg/ml gentamycin. CGNs and DRGs were plated at 2500 cells/well in Eagle's Basal Medium with 10% FBS or SATO components, 20 mM KCL, 33 mM glucose, and 50 U/ml penicillin and streptomycin. After 24 hours in culture, the cells were fixed and permeabilized. Astrocytes, meningeal cells, and fibroblasts were stained with the nucleic acid stain DAPI (Molecular Probes) and CGNs were stained for βIII tubulin. Cell attachment was determined by counting the number of adherent cells in 6 viewing fields (20×) per well (n=4 wells). Neurite extension was measured from digital images using Image Pro software. Data were analyzed by ANOVA using Tukey's method for multiple comparisons of means with p<0.05 considered significant and expressed as mean+/−SEM.

Primary astrocyte, meningeal cell, and dermal fibroblast attachment to surfaces treated with the various conditions is shown in FIG. 13. Cell attachment was significantly lower on covalently immobilized L1-Fc (L1-PDS) relative to fibronectin under all conditions. Furthermore, covalent immobilization of L1-Fc significantly decreased dermal fibroblast cell attachment relative to adsorbed L1-Fc in the presence of serum.

DRG attachment and neurite extension on fibronectin, L1-Fc, and PLL are shown in FIGS. 14 and 15, respectively. DRGs attached equally well to FN or immobilized L1, whereas cell attachment was significantly reduced on adsorbed L1. DRG neurite outgrowth was significantly higher on L1 either in the covalently immobilized or the adsorbed form compared to FN, PLL or the untreated surface controls.

CGN behavior was different on the same set of substrates. Cell attachment was significantly higher on covalently immobilized L1-Fc and adsorbed L1-Fc relative to fibronectin in the presents of serum (see FIG. 16). Neurite extension on covalently immobilized L1-Fc and adsorbed L1-Fc was significantly greater than on fibronectin or PLL, a common culture substrate for neurons (FIG. 17).

The results of our studies suggest that covalent immobilization of L1 -Fc provides biomaterial substrates with a surface that is highly selective for neuronal outgrowth. These materials provide an inhibitory surface for the attachment of other cell types that are frequently encountered in the site of injury or have the potential to colonize a surgical site. The attachment of dermal fibroblasts, astrocytes and meningeal cells to substrates with covalently immobilized L1-FC in the presence of FBS was significantly decreased when compared to their attachment to biomaterials coated with serum fibronectin or PLL. Neurite extension was greater on L1 treated surfaces than that observed on either PLL or fibronectin. These results strongly suggest that the presentation of neuronal cell adhesion molecules by covalent immobilization may be useful in promoting specific neuronal attachment and axonal outgrowth. At the same time, the surface tends to be non-permissive for other anchorage-dependent cells that have the potential to express molecules that are known inhibitors of nerve regeneration.

II. Effect of Varying Filaments Diameter on the Directional Outgrowth of Neurons

To date our cell material interaction studies clearly indicate that surface adhesivity and surface micro-texture are important determinants of adherent neuronal, astrocyte and fibroblastic cell behavior, especially for biomaterials with relatively flat solid surfaces. However, as mentioned in our earlier reports, in cases where a biomaterial takes the shape of a filament or other complex curved geometry an additional parameter may influence cell behavior. In these cases, three variables are potentially at play, namely, surface adhesivity, surface micro-texture and the curvature of the material surface. To understand the relative importance of these factors, we initiated studies that sought to examine the relative contribution of surface curvature on the behavior of adherent cells while maintaining surface adhesivity and surface micro-texture constant. As described previously these studies involve growing neuronal cells, and more recently other accessory cells such as Schwann cells and astrocytes, on filaments of varying diameters and quantifying the orientation of the growing cells or axons relative to the long axis of the filament.

To quantify the directionality of neuronal outgrowth, neurites extending from the cell body were broken into uniform segments that were then evaluated at an angle θ relative to the long axis of the fiber. A histogram was generated that displays the percentage of neurite growing relative to the direction of the long axis where the x-axis was broken into 10 degree segments that ranged from −90 to 90 degrees (FIG. 18). That is, Bin 1 represent the percentage of neurites that grow 90° to the left of the long axis, while bin 18 represents the percentage of neurites that grow 90° to the right of the long axis. Bins 9 and 10 represent the percentage of neurites extending 10° to either side of the long axis. Histograms are generated for various filament diameters.

Filaments melt extruded in diameters ranging from 30 microns to 500 microns were attached to metallic frames, washed in 1% alconox for 10 minutes, and sterilized in 70% ethanol for 1 hr. The filaments were treated with poly-1-lysine (PLL)(50 μg/ml) for 3 hrs, rinsed, and placed in laminin (20 μg/ml) for 1 hr. Purified populations of postnatal day 1 (P1) astrocytes from rats were obtained as described previously. Briefly, cerebral cortices stripped free of meninges were removed, mechanically dissociated with a scalpel, chemically digested, and then triturated. Cells were plated into culture flasks containing Dulbecco's Modified Eagle Medium supplemented with 10% fetal bovine serum (DMEM-FBS) and astrocytes were purified to greater than 98% purity using previously published procedures.

Dorsal root ganglion neurons (DRG) were isolated from P1-P4 rats. Briefly, the spinal column was bilaterally opened from the dorsal side and the spinal cord removed. Individual ganglia were removed, stripped of connective tissue, and placed in buffered solution of trypsin (0.25%) and collagenase (1.33%) for 30 minutes. Following digestion, the tissue was triturated, centrifuged (1000 r.p.m. for 5 minutes) and resuspended in 1 mL of DMEM containing 10 uL of purified 04 antibody and 100 uL of purified rabbit complement (Sigma) for 15 minutes to kill Schwann cells. The cells were counted using a hemacytometer, plated on the filaments at a density of approximately 1×10⁶ cells*ml⁻¹ and incubated for 36 hrs. Mono- and co-cultures were grown in DMEM-FBS with 10 ng/mL 2.5S NGF (Gibco) for 36 hours upon which the cultures were fixed with fresh 4% paraformaldehyde and permeabilized with 0.5% Triton (Sigma) for 3 minutes. Actin cytoskeleton was visualized using rhodamine phalloidin (Molecular Probes). Neurons were identified by staining with antibody against β-III-tubulin (Sigma) and astrocytes were identified by immunoreactivity to glial fibrillary acidic protein (GFAP; Dako). Appropriate fluorescently conjugated secondary antibodies were applied and the samples mounted on slides. Images were taken using a Nikon E600 microscope equipped with epifluorescence and a digital camera (Coolsnap; Roper Scientific). Analysis of cell morphology, cytoskeletal structure, and neurite length was conducted using Image Pro software (Media Cybernetics). Samples were placed in 1% osmium tetroxide and dehydrated. After coating with gold, SEM images were taken along the length of all the fibers. Angle measurements were made as described previously and histogram was generated showing the percentage of neurites that grew at angles relative to the long axis (FIG. 18).

Primary DRG's and astrocytes attached to all of the materials irrespective of radius of curvature or the width of planar substrates studied. Astrocyte coated filaments provided a favorable substrate for DRG neurite outgrowth. Both cell types displayed morphologies and cytoskeletal proteins that were aligned to varying degrees with the long axis of filaments, whereas random organization was observed on planar surfaces (see FIG. 18). The pattern of neurite formation changed as a function of surface curvature from a star like-pattern multi-process morphology seen on the flat surfaces and large diameter filaments, to a more uni- and bipolar morphology on the surfaces with more curvature. The induction of a pseudo-unipolar morphology by filaments was enhanced as a function of the surface curvature. Filaments with a smaller radius resulted in a neurite outgrowth that in general had a single straight process that grew in the direction of the filament long axis. DRG neurites were more directionally biased along the long axis at filament diameters under 300 um in diameter. FIG. 18 provides an example of representative data for 7 substrates including a flat polypropylene surface and filaments of decreasing diameters of from 500 μm to 35 μm. Note that on the flat polypropylene surface the distribution of angles was uniform across all angle measurements indicating that there was an equal probability of outgrowth in all directions. As the surface curvature increases the probability distribution of neurite outgrowth tightens dramatically. There becomes a higher probability of outgrowth in the direction of the long axis of the filament as radius of curvature increases. In addition, the data indicate that there is a minimum filament diameter where the entire outgrowth is constrained to the direction of the long axis of the filament. We intend to model the data to predict the ideal filament size to constrain growth along the long axis. Studies using astrocytes, fibroblasts, and Schwann cells indicate that filament radius of curvature strongly influences the morphology and cytoskeletal organization of the adherent cells.

Additional studies in this general area suggest that substrate-induced cytoskeletal organization can be transferred to additional adherent cell layers, a technique we have labeled “secondary layer patterning”. Filaments coated with astrocytes appear to impart directionality to bound neurons. We believe that our observations can be explained by a mechanotransduction mechanism whereby physical information from the biomaterial surface is imparted to neuron-adhesive ligands on the apical surface of the astrocytes. Our preliminary observations indicate that alignment of the astrocyte cytoskeleton may plays a critical role in generating a pattern or template for directing linear neurite extension. Our studies have shown that in striking contrast to the multidirectional outgrowth of neurons plated on astrocytes seeded on smooth surfaces, neurons growing on an oriented layer of astrocytes are significantly longer, growing in parallel arrays to the direction of the underlying microtopography. This discovery provides a means of promoting ordered tissue growth that may be applied to the repair of target directed axonal pathways, and may be translated into reparative strategies for a variety of other mesenchymally-derived tissues. The implication of this discovery is that biomaterials can be engineered that influence cell behaviors away from the material surface so that they can be used as a template. This approach appears to influence the organization of tissue much like it is believed that a pioneering axon influences the development of a nerve fiber bundle. More importantly, from a commercial standpoint this important discovery may have broad implications for the engineering other types of tissues.

### EXAMPLE 8

Device Development:

A conceptual design is shown in FIG. 19 of a multiple filament NFG implant system and consists of 4 different components including: a luer connector for syringe attachment; a transparent piece of tubing for visualizing filament coating or cell loading by using a stereo magnification; a semipermeable hollow fiber that serves to bundle the filaments as well as isolate the filaments from host inhibitory cells; and a bundle of filaments which may contain genetically engineered L1 expressing or trophic factor secreting cells. We have built a number of different prototypes that utilize a length of biocompatible, tubular, semipermeable hollow fiber made of polyacrylonitrile-vinyl chloride copolymer with an outer diameter of approximately 500 microns. Initial engineering efforts have focused on building prototypes that differ primarily in the number and size of filaments they contain. A number of devices have been assembled with up to several hundred 6 micron diameter polypropylene filaments. The design allows the user to pass a number of different solutions over the surfaces of the filaments without having to handle the device. This allows the user to select the desired surface activation method including type of surface ligand and/or cell type.

The semipermeable membrane (see FIGS. 19& 20) facilitates handling and may prevent colonization of the NFG filaments along the length of the implantation site. The proximal portion of the device is modified to allow insertion in the remaining normal tissue of the rostral side of the cord following either hemisection or contusion injury.

Prototypes seeded with DRG explants and cultured for up to one week revealed extensive DRG outgrowth along the filament bundle with extensive Schwann cell migration up to 7 mm from site of the explant. Schwann cells observed on the filament surfaces displayed a bipolar spindle shaped morphology aligned with the long axis of the filament and served as a substrate for the DRG neurites (see FIG. 21).

### EXAMPLE 9

Effect of Varying Substrate Oriented Microtexture on the Cytoskeletal Organization of Adherent Astrocytes:

Polymeric substrates with an oriented surface microtexture were produced from a template by a heat molding procedure previously described. Six templates of gradually increasing surface roughness were used. The templates were arranged adjacent to one another, so that a single continuous molded culture substrate containing all of the textures could be used in the same culture dish (FIG. 22). In order to determine the precise topographical dimensions of the molded polystyrene, samples were analyzed by atomic force microscopy (AFM). AFM analysis indicated the surface topography was heterogenous in nature (FIG. 23). With six distinctly different surfaces that contained successively deeper grooves and wider distances between major grooves. The groove depths on the substrate surfaces ranged in general from the size of supramolecular protein complex of 20 to 50 nanometers to features of cellular dimension of from 0.5 to 1.8 microns.

The behaviors of primary astrocytes isolated and purified from the cortices of newborn rats were investigated on these materials. Astrocytes were seeded at low densities so as to produce subconfluent layers after two days in culture. Cultures were fixed and stained for the three major types of cytoskeleton: glial fibrillary acid protein (GFAP) for intermediate filaments, actin, and beta-tubulin. In addition, cultures were stained for vinculin, a cytoskeleton-associated protein found at focal contacts. The data is summarized pictorially in FIG. 24. Analysis of stained cultures indicated that astrocyte morphology was dramatically affected by substrate groove depth. On the surfaces with the smallest features, astrocytes maintained the characteristic flattened and well-spread morphology typical of astrocytes in serum-containing culture medium. However, as groove features increased in size, astrocytes gradually became narrow and polarized along the long axis of the underlying. Coincident with the dramatic change in astrocyte shape, the orientation of actin within cells changed from a random crosshatched appearance in well-spread astrocytes to aligned parallel arrays of actin bundles in astrocytes cultured on the deeper-grooved substrates. As expected, the spatial expression of the actin-associated protein vinculin also began to appear in small narrow streaks that extended predominantly parallel to the long axis of the grooves, indicating that aligned astrocytes also formed parallel arrays of integrin-containing focal contacts. A gradual alignment of GFAP filaments and beta-tubulin, although not as striking as actin, was also observed. The pattern of cytoskeletal alignment described above was similar in both subconfluent and confluent astrocyte cultures, and also remained qualitatively similar regardless of the identity of the adhesive protein used to treat the culture substrates (laminin, fibronectin, or poly-L-lysine).

Cell-derived ECM proteins and some cell adhesion proteins, namely NCAM, are known to be attached to and organized by the actin cytoskeleton. At least in the case of ECM proteins like fibronectin, organization appears to be dependent on actin-linked integrin receptors. Because the astrocyte cytoskeleton, as well as sites of focal contacts were found to be aligned as a result of grooves in the culture substrate, we sought to determine whether the spatial expression of ECM and cell adhesion proteins in astrocytes were influenced by culture on substrates with oriented microtexture. The expression of the ECM proteins Cellular Fibronectin (CFN) and the cell adhesion protein Neural Cell Adhesion Molecule (NCAM) were analyzed by indirect immunofluorescence (FIG. 25). Similar to the pattern of cytoskeletal alignment, both CFN and NCAM expression gradually became oriented with increasing microtexture depth of the substrates. CFN expression changed from a characteristic random fibrillar pattern on the least grooved surfaces to elongated streaks that ran parallel to the long axis of the substrate grooves. Similarly, NCAM expression, which was primarily concentrated around the perimeter of the cells on the least textured surfaces, also became elongated in streaks running parallel to the underlying substrate grooves. These results indicate that oriented physical features presented on a biomaterial surface can be transduced and converted, presumably through the actin cytoskeleton, into oriented arrays of cell-adhesive proteins on the surfaces of astrocytes.

### EXAMPLE 10

Effect of Filament Curvature on the Behavior of Adherent Cells:

We also have continued to study the influence of surface curvature on the behavior of adherent neurons, astrocytes, Schwann cells and fibroblasts with both qualitative and quantitative methods. In particular, we have completed our analysis of the effect of filament curvature on the directionality of neurite outgrowth, and have begun a more 37 detailed and complete analysis of the affect of surface curvature on astrocyte shape and cytoskeletal organization. In this quarterly report, we present our qualitative analysis of astrocyte morphology and cytoskeletal organization.

The methodology employed to manufacture the filaments, isolate the cells, seed the cells on the filaments, the subsequent fixation and immunoflourescent methods, and analysis have been described in several previous quarterly reports and are therefore not included here. For these studies, several size classes of polypropylene filaments were used including diameters of: 500 um, 300 um, 200 um, 100 um, 75 um, and 35 um. Breifly, the filaments were washed in 5% alconox for at least 5 minutes and sterilized in 80% ethanol for 30 minutes. A coating of poly-d-lysine (PDL) was applied by soaking the filaments in a 50 ug/ml solution of PDL for at least 1 hour. Subsequently, a second layer of laminin was added using a 20 ug/ml solution in PBS for 1 hour. Each step was preceded by a 5 minute wash in sterile PBS. The cells were fixed with paraformaldehyde, permeablized with triton, and stained for actin, vimentin, and GFAP.

Distinct morphological differences were observed as a function of surface curvature. The astrocytes grown on the 500 um filament were well spread with the cytoskeleton exhibiting no apparent polarity. Astrocytes shape was generally polygonal. On the 300 um diameter filaments, some of the astrocyte population display a more elongated morphology, with many others adopting a polygonal morphology. The elongated cells contain an actin and GFAP filaments that exhibit some bias along the long axis of the filament, but the behavior is not striking. On the 200 um filaments, there is approximately the same number of spread and elongated astrocytes. However, the actin and GFAP filaments alignment distinctly favors the main axis of the fiber. On the 100 um filaments. The elongated morphology is preferred, but some polygonal cells are still present. Many of the astrocytes display a bipolar morphology with long, spindlely processes. This morphological class of cells is not found on filaments of larger diameters. At a filament size of 75 um, nearly all of the cells display the bipolar, highly elongated morphology with their actin and intermediate filament cytoskeleton highly polarized. Finally, at 35 um most of the adherent astrocytes display a marked elongated morphology and appear to stain less intensely for GFAP. These studies are still underway and a more quantitative analysis appear in future reports as our studies in this area progress.

### EXAMPLE 11

In Vivo Studies:

Based on the results of studies described in the preceding sections, we have selected polypropylene as the base material for the in vivo evaluations of the substrates for the directed growth of injured axons following spinal cord injury in adult rats. The initial studies were designed to examine: 1) biocompatability of the polymers (that is, can the biomaterials integrate with the host spinal cord tissue, or will the host generate a “foreign body” response to placement of the material into adult spinal cord tissue, i.e., is there histological evidence of astrogliosis and invasion by neuroimmune infiltrates?); 2) different surgical methods of rod placement into spinal cord tissue (that is, can the NFG constructs be implanted in a regionally specific manner in the spinal cord to address the response of particular axonal populations?); and, 3) the optimal methods for processing polymer implanted tissue histologically. Several studies have been conducted.

For these studies, female Fischer 344 rats were implanted with both large diameter (130 micron) and small diameter (70 micron) NFG filaments into the dorsolateral thoracic spinal cord. Each rat received a laminectomy at T9. The dura was exposed and opened, and a small slit was made into the dorsal surface of the spinal cord cord lateral to the midline using the tip of a #11 scalpel blade. The slit provided an opening into the spinal dorsal spinal cord, into which were placed either 1 large diameter filament (animal's right side) or 3-to-4 small diameter filaments (animals left side). Both sizes of filament were non-treated with surface coatings or ECM molecules to establish baseline material host reactivity. The rods were inserted using jewelers' forceps, in the longitudinal plane, with care to minimize damage to the spinal cord. After implantation, the filaments could not be observed with magnification from the surface of the cord. The overlying muscle layers were sutured and the skin incision closed with stapled. Only postoperative observation was required. Following a two week survival period, the polymer-implanted animals were transcardially perfused with ice cold buffered saline (0.1 M phosphate buffered saline (PBS)) followed by 4% paraformaldehyde (also in PBS). The spinal columns were removed and placed into a 4% paraformaldehyde overnight at 4° C. The following day, the cords were removed from the vertebral columns and placed into a 30% sucrose solution for 3 days. The spinal cords were then cut on a cryostat.

Tissue Processing & Histology:

Because of the differing mechanical characteristics of the biomaterial scaffolding and the host tissue at the site of implantation, novel methods of histological evaluation have to be developed. Along these lines, several histological methods are being evaluated for examining the host biomaterial interface including cryostat sectioning for sections less than 15 microns in thickness and Vibratome for thicker sections.

Tissue sections were processed immunohistochemically, either as free-floating sections or directly mounted onto slides. Sections have been analyzed with antibodies against: neurofilaments (to examine any overt axonal response to the rod placement), Substance P (to determine response from nociceptive sensory afferent axons from the dorsolateral fasciculi), GFAP (for reactive/non-reactive astrocytes), ED-1 for activated macrophages, OX-42 for microglia, CS-56 (a general marker for the family of chondroitin sulfate proteoglycans (CSPGs)) as a marker of reactive matrix formation and for possible inhibitory molecule deposition, and specific CSPG core protein markers Neurocan, Phosphacan and NG2 proteoglycan. Sections have also been processed with thionin to examine overall cellular response to the implanted material. Example photomicrographs are shown in FIGS. 5-11, which show transverse sections through the spinal cord at the level of the filament implants. The filament material itself is lost in the processing of the tissue for sectioning, and the position of the filament shows as a circular space in the tissue.

Based on histological data obtained from Nissl staining, there did not appear to be a dramatic cellular response to the implanted rod. A layer several cell layers thick formed around the implants by two weeks post implantation. The size of this layer appears to correlate with the diameter of the rod. ED-1 and OX-42 labeling indicate that macrophages/microglia compose this cellular layer or at least compose a major component of it. A host gliotic response, as indicated by GFAP-staining, appears to be minimal to non-existent by two weeks post implantation. Host axons (NF- and Substance P-immunoreactivity) were observed intimately associated with the cellular layer surrounding the polypropylene filaments, but few were observed associated with the surface of the implant. The host material interfacial zone was also immunoreactive for several putative inhibitory proteoglycans including a general CSPG marker (CS-56), neurocan, phosphacan and the NG2 proteoglycan. The results of our initial biocompatibility and handling studies indicate that the material can be easily manipulated and placed into discrete areas of the adult rat spinal cord.

The following study details the integration of multifilament devices into the injured spinal cord. In particular, we have been concerned to confirm that pre-filling of the constructs with perinatal (postnatal day 5) astrocytes provides an improved environment within the device. We have also begun to examine the cellular nature of the tissue within the constructs in more detail.

In vivo studies we continued to focus in four general areas: biocompatibility or host response to the NFG biomaterials; the development of a technology to control the placement of the small diameter NFG filaments into precise locations in the spinal cord; packaging issues relating to sterility and transport of the delicate protein coated materials from one site to another; the development of histological protocols for assessing differences in the tissue response to the different types of surface treated implants. Two sizes of uncoated polypropylene filaments were implanted in rat spinal cord −137 μm and 67 μm diameter. Small coronal incisions were placed in the dorsal surface of the spinal cord to facilitate filament insertion. Filaments were directed along the longitudinal axis of the spinal cord, in tracts paralleling the major direction of axonal travel in the rostro-caudal direction. Individual filaments were handled with fine jeweler's forceps. Animals were sacrificed two weeks after placement of the filaments and were transcardially perfused with 4% paraformaldehyde to allow performance of immunocytochemical analysis.

Frozen sections of the spinal cords were cut at 35 μm thickness. Subsequent sections were stained or immunolabeled as follows:

Nissl stain (thionin)

Immunolabels:

| LABEL | DETECTS |
|---|---|
| Neurofilament (RT97) | All Axons |
| Calcitonin Gene- | Sensory Axons |
| Related Peptide | |
| Substance-P | Nociceptive Sensory |
| Axons | |
| Glial Fibrillary | Astrocyte Reactions |
| Acidic Protein | |
| (GFAP) | |
| OX-42 | Microglia |
| ED-1 | Activated |
| Macrophages | |
| Chondroitin Sulfate | Extracellular Matrix |
| Proteoglycan (CS-56) | |
| Neurocan | Extracellular Matrix |
| NG2 | Extracellular Matrix |

Examples of staining with these antibodies are provided in the attached figures. Microscopic analysis of implanted filaments suggests that the materials are well tolerated by the host and elicit a minimal inflammatory response, characterized by both an acellular and a cell-reactive layer composed of GFAP positive astrocytes, Ox-42 positive microglia and related cell types including meningeal cells and monocytes. Immunolabeling for neurofilament, CGRP and Substance-P showed no enhancement of axonal growth along uncoated filaments, as expected. GFAP labeling reveals a mild glial reactivity along the course of the implanted filament, but no massive glial response. OX-42 and ED-1 labeling reveal a modest but possibly significant host cellular microglial and macrophage response to the presence of the filament. The most significant differences in host reaction appear to be associated with regional differences within the spinal cord cross section. That is, there was a greater response in white matter compared to that observed for the same material passing through gray matter.

These experiments involved the use of the multi-filament device, and concerned examining the handling and cellular response to the device with and without the addition of adherent accessory cells, including fibroblast, Schwann cells and astrocytes. The goal of accessory cell attachment is to make the surfaces within the device more conducive to neurite extension and also vascularization.

These experiments have been carried out in dorsal hemisection lesions of the thoracic spinal cord, by implanting the construct into the space left by the aspiration lesion technique (FIG. 26). The aspiration lesion was made slightly shorter than the length of the implant, so that the ends could be interfaced with the intact tissue on either face under slight pressure. Similar experiments were performed using weight drop contusion injuries of the spinal cord. In this case the injury site was re-exposed at 3 weeks post injury, a midline myelotomy performed, to open up the lesion cavity, and the multifilament device could be inserted into this space, again with slight pressure on the interface at either end of the device. The cord was closed over the device, and integrated well into the tissue (FIG. 27). Histological analysis of multifilament devices showed minimal reaction from the tissue.

Based on the results of our earlier experiments with single filaments, the multi-filament devices used in the contusion injury were pre-filled with postnatal astrocytes in vitro. These devices showed good integration, and the interstices between the filaments were well filled with cells at the time of sacrifice, 3 weeks following implantation. The cellular contents of the devices included apparently successful vascularization (FIG. 27 B). These devices and surrounding tissues are now under more detailed examination with immunocytochemistry.

The experiments performed have addressed the following aims:

1) To examine the effects of a variety of anti-inflammatory approaches on the integration of polypropylene filaments within the injured and uninjured spinal cord parenchyma.

2) To examine the cellular response to polypropylene filaments with different cellular coatings, implanted in the spinal cord, in dorsal hemisection lesions, spinal cord contusion lesions, and intact spinal cord parenchyma.

3) To determine whether axonal regeneration after spinal cord injury can be promoted by a combination of delivery of growth-orienting polymer rods and growth-promoting neurotrophic factors, delivered by genetically modified cells.

Anti-Inflammatory Approaches:

Implantation of filaments into otherwise uninjured thoracic spinal cord was used to test the ability of anti-inflammatory approaches to reduce the cellular reaction of the tissue to the engineered filaments, which was noted in earlier reports. Methylprednisolone sodium succinate, cyclosporine, and FK506 have been examined separately and in combinations, with both systemic delivery and intrathecal infusion. At this time, qualitative data on the success of these approaches is available, but ongoing quantitative analysis of the reactivity of the tissue will be presented in a subsequent report.

In general, the least reactive of the implanted filaments were those coated with the surfactant Pluronic, F-108. The reactivity of this material was reduced even further with anti-inflammatory approaches, particularly by local infusion of methylprednisolone. In the best cases, there was no visible macrophage or microglial involvement in the tissue reaction around the filaments. However, in all cases a thin layer of cells coated the surface of the fiber, probably composed of meningeal elements. This layer produced a separation between the filament surface itself and the surrounding central nervous system parenchyma. We therefore concluded that simply suppressing the inflammatory response is unlikely to be sufficient to produce a true interface of the engineered biomaterials with the central nervous system environment in vivo. This leads us to an even stronger interest in the potential for cellular coating to increase the integration of the implanted filaments.

Cell Coating and Growth Factor Release:

These goals continue to address the central question of the project: whether synthetic polymer filaments can provide a conducive growth and alignment substrate to injured adult spinal cord axons in vivo.

To test the hypothesis that growth factors can attract injured axons to the filament surface, primary rat fibroblasts or Schwann cells were genetically modified to produce and secrete the potent nervous system growth factors Nerve Growth Factor (NGF) or Glial Cell-Line Derived Neurotrophic Factor (GDNF). These genetically modified cells were attached to polypropylene filaments provided by the University of Utah, and were then implanted into Fischer 344 rats with mid-thoracic spinal cord dorsal hemisection lesions.

Primary Fischer 344 rat fibroblasts or Schwann cells were genetically modified to produce and secrete human NGF. In vitro, prior to implantation, these cells secreted approximately 10-20 ng human NGF/10⁶ cells/day into the conditioned medium. This represents levels of NGF production approximately 500-fold above physiological levels. Control cells were either not transfected, or expressed the reporter gene Green Fluorescent Protein (GFP). Genetically modified fibroblasts or Schwann cells were bound to filaments by placing the filaments in petri dishes containing the genetically modified cells. After approximately 72 hours in vitro, cells spontaneously associated with the filament surface and, as has been shown in previous progress reports for a range of cell types, they tended to orient along the longitudinal axis of the filaments.

A total of 24 neurotrophin-bearing (12 NGF, 12 GDNF) and 24 control filaments (12 GFP rods, 12 uncoated rods) were examined in adult Fischer 344 rats with dorsal spinal cord hemisection. Filaments from each experimental group were examined after either 2 or 4 weeks in vivo.

Findings: The association of axons with the filaments was substantially enhanced by coating the filaments with neurotrophin-secreting cells. Addition of NGF-secreting fibroblasts appeared to draw axons through putatively inhibitory cellular elements and into close association with the rod surface. Addition of GDNF-secreting fibroblasts brought substantially enhanced numbers of axons into the region of implanted filaments, but not as close to the filament surface as the NGF-secreting cells (FIG. 28).

Our studies in this area have focused on analyzing the applicability of the method on various biomedically relevant hydrophobic materials. As reported earlier, the method appears to be capable of titrating ligand density as evidenced by ELISA and is effective in regulating neurite outgrowth by varying substrate ligand concentration (see FIG. 29 for laminin & FIG. 30 for fibronectin). It appears to be effective as long as the material is sufficiently hydrophobic or possesses a static contact angle of greater than 75 degrees, and providing that sufficient time is allowed for adsorption to the surface.

During the past quarter, we extended our studies in this area by comparing the bioactivity of FN to the surface concentration of FN. For these studies, we studied dorsal root ganglion neuron attachment and neurite outgrowth in the presence of NGF. ¹²⁵I-labeled FN was applied directly to polystyrene surfaces by adsorption or indirectly by immobilization to a surface coating containing end-group activated polyethylene oxide (PEO)-containing triblock surfactants, Pluronic® F108. The bioactivity results were also compared to ELISA assays of the same treatments. We provide evidence that FN immobilized via activated F108 coatings support approximately eight times more neurite promoting bioactivity when compared to FN directly adsorbed to polystyrene in the presence of serum.

The terminal hydroxyl groups of the PEO chains of Pluronic® F108 (“F108”, BASF Corporation) were modified with PDS groups. Briefly, 4 g F108 was reacted with 0.33 g 4-nitrophenol chloroformate in 16 mL benzene for 24 hours. Nitrophenol-activated F108 was recovered by precipitation with ethyl ether. Pyridyl diethyl ammonium (PDEA) was prepared by reaction of 1.13 g mercaptoethylamine-HCl and 6.74 g 2-2′ dithiopyridine in 32 mL methanol and 1.2 mL glacial acetic acid for 30 minutes. The PDEA product was recovered by precipitation with ethyl ether. To prepare activated F108 (or F108-PDS), 2 g nitrophenol-activated F108 was reacted with 1.2 g PDEA in 12 mL methanol with 2.5 mL triethylamine overnight. The F108-PDS product was recovered by dialysis (1000 MWCO) against distilled water for 48 hours then freeze-dried for storage. To characterize the efficiency of PDS incorporation, F108-PDS was dissolved in distilled water at a concentration of 0.33 mg/mL and reduced with 10 μL of dithiothreitol (DTT, Sigma) for 1 hour. The molar concentration of the pyridyl leaving group was determined by A₃₄₃ with a molar extinction coefficient of 8080 M⁻¹ cm⁻¹.

Bovine fibronectin (Gibco BRL) was thiolated by reaction with N-succinimidyl 3-(2-pyridyldithio) propionate (SPDP, Pierce). Briefly, 11 μL of 5 mM SPDP freshly dissolved in DMSO was added to 1 mL of 1 mg/mL fibronectin and mixed for 1 hour (25×molar excess of SPDP). The 2-pyridyl-disulfide-modified protein was separated from excess SPDP on a PD-10 column (Pharmacia) using 1×PBS (pH 7.4) to elute the protein fraction. Fractions containing protein were pooled and mixed with 10 μL of 25 mM DTT for 1 hour to reduce the 2-pyridyl disulfide groups. Absorbance readings at 280 nm and 343 nm were performed, to determine the molar concentration of the protein and the pyridyl leaving group, respectively. The thiolated fibronectin was separated from excess DTT by passage over a PD-10 column using 0.1M sodium phosphate buffer with 5 mM EDTA (pH 6) as the elutent. The final protein concentration was determined from the A₂₈₀ against a standard curve for fibronectin concentration.

Dorsal root ganglion neurons (DRG) were prepared from postnatal day 1 rats. Dorsal ganglia were individually removed from the spinal column and placed in a dish of ice-cold L15 medium (Gibco). The nerve roots were stripped, and the remaining ganglia were placed into a solution of L15 containing 1.33% (w/v) collagenase (Sigma) for 45 minutes. The collagenase-digested suspension was centrifuged at 600 g for 3 minutes then placed into a solution of Dulbecco's Modified Eagle's Medium (DMEM; Gibco) containing 0.25% trypsin (w/v) and 0.1% (w/v) DNase (Worthington) for 30 minutes.

The digested tissue was again centrifuged at 600 g for 3 minutes then resuspended in a small volume of DMEM containing 0.1% DNase. The suspension was triturated with fire-polished Pasteur pipettes of decreasing bore diameter, centrifuged at 1000 g for 5 minutes, and resuspended in 1 mL of DMEM containing 04 antibody[Sommer, 1981 #30] (1:100) and 10% rabbit complement (Sigma) for 30 minutes. The O4 complement kill is a purification step to remove contaminating Schwann cells from the suspension. The suspension was diluted to 10 mL with DMEM and centrifuged at 1000 g for 5 minutes followed by 3000 g for 1 minute. Cells were resuspended in DMEM-F12 (Gibco) supplemented with defined components, 10 ng/mL 2.5S Nerve Growth Factor (NGF, Gibco), and plated at appropriate density.

Ninety-six well polystyrene (NUNC) plates were adsorbed overnight with varying solution concentrations of fibronectin (further referred to as PS-FN). Cells were plated after rinsing three times with PBS. For immobilization, 96 well polystyrene plates were adsorbed overnight with varying ratios of F108-PDS:F108, maintaining a 1% (w/v) final concentration (further referred to as F108-FN). After rinsing three times with distilled water, the plates were incubated overnight with 100 μg/mL thiolated fibronectin in PBS. After incubation with protein, the plates were washed three times with PBS prior to cell seeding.

Ninety-six well plates with adsorbed or immobilized fibronectin were initially blocked with 3% (w/v) bovine serum albumin (BSA) in PBS for 1 hour to prevent non-specific binding of antibodies. Each well was treated with polyclonal rabbit anti-bovine fibronectin antibody (Chemicon, 1:1500) for 1 hour. Plates were thoroughly rinsed in PBS and then treated with HRP-conjugated goat anti-rabbit IgG antibody (Chemicon, 1:1500) for 1 hour followed by another washing and QuantaBluÔ Fluorogenic Peroxidase Substrate (Pierce) for 20 minutes. The reaction was terminated with stop buffer provided by the manufacturer. Control wells without any protein were also examined. The fluorescent product of the reaction was measured in relative fluorescence units (RFU) using a fluorescent plate reader (Cytofluor II, Perseptive Biosystems) with 360 nm excitation and 460 nm emission filters. All ELISA data are presented with the control background RFUs subtracted. A minimum of 4 wells was measured for each experimental condition.

Fibronectin was labeled with ¹²⁵I by the Chloramine-T method to a specific activity of 0.52 mCi/mg. Individual wells were cut from 96-well polystyrene plates (Nunc) and cleaned with ethanol and water. For adsorption studies, 100 μl of ¹²⁵I-fibronectin was diluted in PBS to 100, 50, 10, and 1 μg/ml and adsorbed to individual wells (n=4/condition) overnight. For immobilization studies, ¹²⁵I-fibronectin was diluted to 1 mg/ml, thiolated as described above, and collected in 0.1 M sodium phosphate buffer with 5 mM EDTA (pH 6). Polystyrene wells were treated overnight with 1% (w/v) solutions containing various ratios of modified to unmodified surfactant at 100:0,75:25,25:75, and 17.5:82.5 for F108-PDS:F108, respectively. Following the incubation period, each well was rinsed with distilled water, and treated overnight with thiolated ¹²⁵I-fibronectin [100 μg/ml] (n=4/condition). All surfaces were rinsed four times with PBS, placed in scintillation vials, and counted in a Packard Minaxi Gamma Counter.

Approximately 300 freshly dissociated P1 rat DRG neurons were seeded into individual wells of each prepared 96 well plate. DRG neurons were cultured in either serum-free medium or serum-containing medium. Serum-free medium consisted of DMEM-F12 supplemented with defined components, gentamycin, and 10 ng/mL mouse 2.5S NGF. Serum-containing medium (DMEM-FBS) consisted of DMEM-F12 supplemented with gentamycin, 10% fetal bovine serum, and 10 ng/mL mouse 2.5S NGF. Cells were cultured for 24 hours, rinsed, fixed in 4% paraformaldehyde and processed for immunostaining.

For immuno-analysis, neurons were treated with 0.5% Triton-X-100 for 5 minutes after paraformaldehyde fixation. Wells were rinsed with staining medium (Hanks balanced salts solution with 0.05% (w/v) sodium azide, 5% donor calf serum, and buffered to pH 7.4 with HEPES), and primary antibody against neurofilament (Sigma) or βIII tubulin (Sigma) (diluted 1:100 in staining medium) was applied for 1 hour. Wells were rinsed again with staining medium and the appropriate Texas Red-conjugated secondary antibody was applied for 1 hour. Following the secondary antibody, wells were rinsed and filled with PBS. Images of immunostained neurons were captured using a digital camera attached to a Nikon inverted microscope equipped with epifluorescent illumination. Attachment efficiency was quantified by counting the total number of neurons in each treated well of a 96-well plate. Neurite outgrowth was measured using Image Pro Plus (Media Cybernetics) image analysis software following calibration. A minimum of 200 cells from at least 3 independent experiments was analyzed for each experimental condition.

Cell Attachment to Polystyrene: Adsorbed vs. F108-Coupled Fibronectin

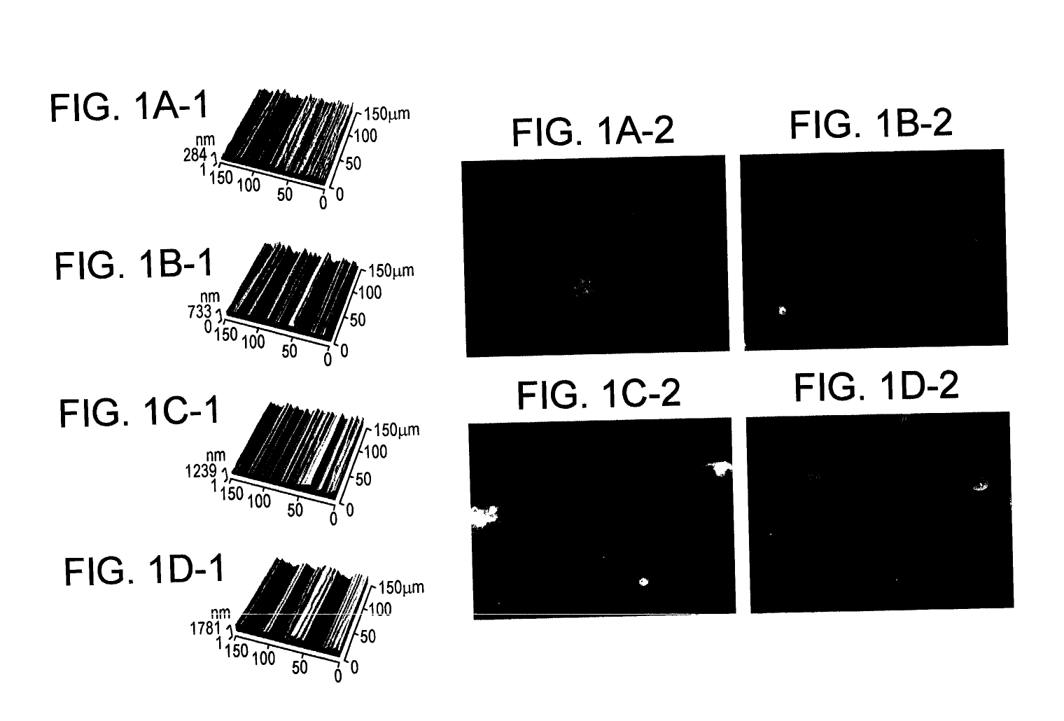

DRG cell attachment at varying concentration of soluble FN directly adsorbed to polystyrene in the presence and absence of serum containing media is shown in FIG. 1. A significant amount of cell attachment was observed on untreated polystyrene (PS), that is, in the absence of exposure to FN treatment (0 soluble FN concentration), indicating that DRG's do not require FN for binding to PS substrates. We believe the DRG attachment in the absence of FN are most likely mediated by proteins attached to the cell surface membranes of the DRGs. Since we were interested in measuring FN induced bioactivity, we chose to subtract the cell binding activity when no FN was applied to the surface. The original data appears in the upper panel of FIG. 1. The corrected data appears in the lower panel. DRG's attached over the entire range of FN surface treatments both in the presence and in the absence of media containing serum proteins. Maximal attachment was not significantly different under either media condition. Under serum free conditions, cell attachment increased gradually reaching a maximum at 1 μg/mL and rapidly declined as the solution concentration of FN applied to the surface increased. This was in contrast to the results obtained in the serum containing media, where cell attachment gradually increased in response to applied FN reaching a maximum at a 5-fold higher concentration (5 μg/mL) that leveled or plateaued as higher concentrations of FN were applied. Significantly, less cell attachment was observed on untreated PS (0 soluble FN concentration) in the presence of serum containing proteins, perhaps indicating that such serum proteins as albumin may inhibit DRG attachment under these growth conditions.

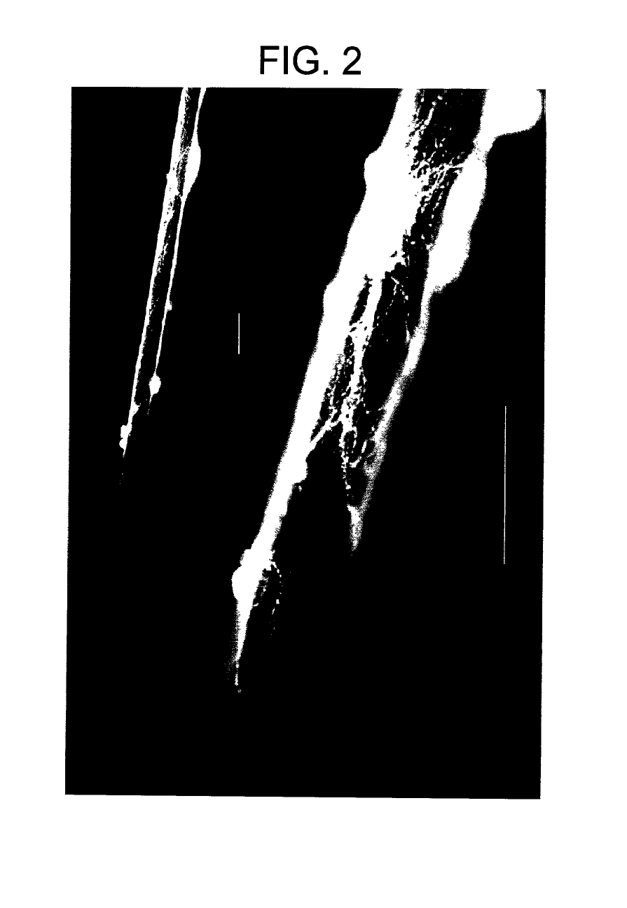

DRG cell attachment to FN immobilized via the surfactant coating (F108-FN) is shown in FIG. 2. Little cell attachment was observed on surfactant treated surfaces in the absence of FN. DRG's attached over the entire range of treatment conditions, gradually increasing as the ratio of F108-PDS increased. Values for cell attachment in serum-free and serum-containing media were not significantly different. The maximal level of attachment for FN immobilized through the surfactant coating was not significantly different from the maximal levels observed when FN was adsorbed directly to polystyrene.

DRG Neurite Outgrowth on Polystyrene: Adsorbed vs. F108-Coupled Fibronectin

DRG neurite outgrowth as a function of FN directly adsorbed to polystyrene with and without serum containing media is shown in FIG. 32. A significant amount of neurite outgrowth was observed on native polystyrene (PS) in the absence of FN treatment (0 soluble FN concentration), indicating that DRG's did not require FN for neurite outgrowth on such PS substrates, a condition that was most likely mediated by proteins attached to the DRGs that nonspecifically bound to the hydrophobic PS surface. Since we were interested in measuring FN induced neurite outgrowth, we chose to subtract the level neurite outgrowth when no FN was present from the data. The original data appears in the upper panel of FIG. 33, whereas corrected the data appears in the lower panel. Neurite outgrowth was observed over the entire range of surface treatments in the presence and in the absence of media containing serum proteins. Maximal outgrowth was not significantly different under either condition reaching a little over 200 microns in length. The pattern of outgrowth, however, as a function of the surface treatment was different (lower panel). Under serum free conditions, neurite outgrowth increased gradually reaching a maximum at 10 μg/mL and declined as the solution concentration of FN applied to the surface increased. This was in contrast to the results obtained in serum containing media, where neurite outgrowth gradually increased reaching a maximum that was sustained as higher Fn concentrations were applied. Significantly, less neurite outgrowth was observed on untreated PS (0 soluble FN concentration) in the presence of serum containing proteins, suggesting that serum proteins, perhaps albumin, may inhibit neurite outgrowth under these growth conditions.

DRG neurite outgrowth to FN immobilized via the activated surfactant coating (F108-FN) is shown in FIG. 34. No neurite outgrowth was observed in the absence of FN treatment so it was not necessary to correct the data. These results suggest that the PEO rich surface coating most likely prevented non-specific protein binding of proteins attached to the DRGs. On the activated surface coating neurite outgrowth was observed over the entire range of treatment conditions. Outgrowth gradually increased as the ratio of activated surfactant (F108-PDS) increased to a maximum of approximately 400 microns, a 2-fold increase over the maximal neurite outgrowth obtained by FN adsorption. DRG neurite outgrowth was not significantly different in the two media conditions.

Analyses of Surface Bound Fibronectin

ELISA and radiolabeling methods were used to assess substrate bound FN levels. Thiolation of FN did not alter its antigenicity as determined by comparing the antibody binding behavior of adsorbed native FN and batches of the thiolated molecule (data not shown). Our thiolation procedure introduced approximately 8 thiol groups per FN molecule.

The results of the ELISA studies are shown in FIG. 35. The upper panel shows the relative increase in surface bound FN applied directly to the polystyrene substrate by increasing the solution FN concentration up to 100 μg/ml. Detection above background levels was observed at 0.01 μg/ml. Bound flourescence gradually increased as a function of applied FN, reaching a plateau at 1 μg/ml, which was sustained up to 100 μg/ml. For chemical immobilization through the activated surfactant coating (lower panel), treatment was carried out by exposing the surface to varying ratios of activated (F108-PDS) to unactivated surfactant (F108) and then exposing the treated substrates to 100 μg/ml of soluble FN. Substrate bound FN increased as a function of increasing treatments with F108-PDS reaching a plateau at 60% F108-PDS, which was sustained up to 100% activated surfactant. Taken together, the ELISA assays shown in FIG. 5 indicate that both methods allowed titration of surface bound FN.

To compare the absolute amount of FN introduced to PS surfaces by both methods, we used radiolabeled FN and incubation conditions similar to those used for the ELISA assays. Approximately four-fold more ¹²⁵FN was maximally bound to PS by absorption (1.24±0.10 μg/cm²) than through coupling through activated F108(0.30±0.06 μg/cm²). The surface density at 10 μg/ml FN adsorption was not significantly different from the values obtained at levels of activated surfactant above 17.5%. Interestingly, this level of FN surface density is in the range of published values predicted for FN monolayer formation. Also of interest is the fact that surface bound FN immobilized through F108 displayed a gradual saturation behavior at only a slightly higher but statistically insignificant increase, whereas the amount of surface adsorbed FN continued to increase suggesting that multi-layering was taking place.

Correlation Between FN Bioactivity and Surface Ligand Density

Taken together, our results indicate that FN immobilized to PS through the activated surfactant coating supported significantly greater bioactivity that did FN applied by adsorption from solution. When compared at similar surface densities, the two surface treatments differed significantly in bioactivity (FIGS. 36 & 37). In general, on substrates adsorbed with FN, cell attachment (FIG. 36) and neurite outgrowth (FIG. 37) declined with an increase in substrate FN surface density, suggesting perhaps that conformational changes were taking place leading to decreases in protein bioactivity. In contrast, on FN tethered via F108, cell attachment (FIG. 36) and neurite outgrowth (FIG. 37) increased with increasing surface FN density. Interestingly, at almost identical surface concentrations, adsorbed FN was less bioactive in the presence of serum containing media than FN coupled to F108 for promoting neuronal attachment (FIG. 36) and for promoting neurite outgrowth (FIG. 37).

II. The Influence of Surface Curvature on Neurite Outgrowth

During development, a number of cues are presented that direct the orderly development of tissues in a multicellular organism so that the final architecture of the organism is achieved in a stereotyped manner. The nervous system begins to exhibit a sophisticated structure in the form of a neural scaffold composed of pioneering axons early in development in order to achieve its final complexity. The roles of soluble factors as well as cell and matrix bound molecules in the development of this structure have been extensively studied. Nerve fascicles that develop from early pioneering axons suggest that substrate geometry may be an important early determinant of mature neural architecture. Over the past year we have been examining the importance of substrate curvature on the behavior of isolated dorsal root ganglion cells and astrocytes.

For these studies, DRG neurons from P1-P3 rats were cultured on synthetic filaments of varying diameters (data shown in last progress report). Briefly, as filament diameter decreased, axon segments exhibited a more directionally oriented morphology. Following on from these results, a model was formulated based on a hypothesis that cytoskeletal stiffness is an important regulator of cell behavior on curved substrates. Our data suggest that the mechanical properties of the DRG axon limit its ability to bend on substrates exceeding a critical surface curvature.

### Construction of Model

Probability density functions were built using the Boltzman distribution (FIG. 9—color panel), which assumes maximization of entropy is involved in the process creating the behavior, a distribution frequently observed in natural phenomena. An energy term was developed based on the mechanical strain energy needed to bend the cytoskeleton that consisted of a bundle of filaments with a defined bending stiffness and length. Here we assumed that the microtubule bundles in the axon would be of primary importance. Lambda is a lumped parameter (Γ=nbL) that includes the cytoskeletal element bending stiffness (b), the number of filaments in a bundle (n), and the length of the bundle (L).

This parameter was varied using a nonlinear fitting algorithm (IGOR; Wavemetrics) in order to fit the model to the data. A good fit to all the individual distributions was achieved using one Γ value (Γ=6.36E-7 Nm²). Various exponential curves fit to the maxima of each of the probability distributions as a function of increasing fiber radius is displayed in the color FIG. 38. The ability for the model to capture the behavior of the distributions for all filament sizes suggests that the mechanical properties intrinsic to the neuron remain relatively constant. If bundles of microtubules in the axon are assumed to be the transduction elements, then the Γ constant corresponds to a bundle of 40 tubules with a bending stiffness of 2.2E-23 Nm and a bundle length of 2.9939 um, values consistent with predicted axon microtubule structures. Using the length constant, one can calculate a critical fiber radius (120.1 um) below which the intrinsic stiffness of the bundled microtubules begins to divert the orientation of growing axons away from a circumferential path and towards the axis of the filament.

These mechanical properties depend on the gene expression patterns of the nerve cell and could represent intrinsic properties that differ between different classes of neurons. Neurons that are found in long straight axonal pathways may have a stiffer cytoskeleton than neurons that form short highly curved pathways. We know for the nervous system that the initial structure is laid down by pioneering axons and glia. Later arriving neural cells and their processes are built upon this initial scaffold. The use of curved geometry during development may facilitate the need for the complicated gradient cues once an initial scaffold is laid down. The intrinsic mechanical properties of the differentiated cell restrict its possible behaviors. These types of guidance cues would be easier to implement over the larger distances present in late development than gradient cues and may represent an important guidance mechanism utilized in late development. Studies in progress are examining the influence of substrate curvature on glial development. Preliminary results suggest that highly curved substrates may promote a radial glial phenotype (FIG. 10—see GFAP+(green) astrocytes with a filamentous morphology).

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

What is claimed is:

1. A device for the propagation of tissue comprising a bioartificial composite comprised of a substrate having at least one surface capable of the reception and growth promoting retention of a cellular preparation, and a first layer of adherent cells disposed on said surface, said first layer prepared from said cellular preparation, the cells comprising said first layer having cytoskeletal elements aligned uniformly, wherein said bioartificial composite acts as a template to accept a second layer of cells upon said first layer, said second layer comprising an organized layer oriented in the direction of said first layer, wherein said substrate has at least one surface defined by a critical surface curvature and/or topography.

2. The device of claim 1 wherein said substrate has at least one cell accepting surface defined by an oriented surface roughness of at least 200 nm root mean squared.

3. The device of claim 1 wherein said substrate has at least one cell accepting surface defined by a surface curvature of equal or greater than 0.016 microns⁻¹.

4. The device of claim 1 wherein said substrate defines a repeating surface structure.

5. The device of claim 1 wherein said bioartificial composite possesses an over all non-planar shape.

6. The device of claim 1 wherein said substrate is coated with a biocompatible, growth promoting preparation, which preparation minimizes non-specific protein binding and optimizes attachment of said cells.

7. The device of claim 6 wherein said preparation is selected from the group consisting of surfactants, cell adhesion molecules, polycations, cell growth factors, and mixtures thereof.

8. A device for the preparation of implantable tissue comprising a bioartificial composite comprised of a substrate having at least one surface capable of the reception and growth promoting retention of a cellular preparation, and a first layer of adherent cells disposed on said surface, said first layer prepared from said cellular preparation, the cells comprising said first layer having cytoskeletal elements aligned uniformly, wherein said bioartificial composite acts as a template to accept a second layer of cells upon said first layer, said second layer comprising an organized layer oriented in the direction of said first layer, wherein said substrate has at least one surface defined by a critical surface curvature and/or topography.

9. The device of claim 8 wherein said substrate has at least one cell accepting surface defined by an oriented surface roughness of at least 200 nm root mean squared.

10. The device of claim 8 wherein said substrate has at least one cell accepting surface defined by a surface curvature of equal or greater than 0.016 microns⁻¹.

11. The device of claim 8 wherein said substrate defines a repeating surface structure.

12. The device of claim 8 wherein said bioartificial composite possesses an over all planar shape.

13. The device of claim 8 wherein said bioartificial composite possesses an over all non-planar shape.

14. The device of claim 8 wherein said substrate is coated with a biocompatible, growth promoting preparation, which preparation minimizes non-specific protein binding and optimizes attachment of said cells.

15. The device of claim 14 wherein said preparation is selected from the group consisting of surfactants, cell adhesion molecules, polycations, cell growth factors, and mixtures thereof.

16. The device of claim 8 wherein said bioartificial composite is defined by at least one filamentous substrate.

17. The device of claim 8 wherein said bioartificial composite is defined by at least one cylindrical substrate.

18. The device of claim 17 wherein said substrate has a diameter of less than 300 μm.

19. The device of claim 8 wherein said substrate of said bioartificial composite further defines an axially aligned surface topography, and is coated with cell attachment molecules; and a layer of cells attached to said molecules, which cells are adapted to undergo morphological rearrangement to align with the long axis of said substrate; and at least one second cell layer of different cells that attached to the free upper surface of said first layer, which is also adapted to undergo the same said morphological rearrangement.

20. The device of claim 19 wherein the morphological rearrangement of said first layer of cells which comprises the bioartificial composite is promoted and effected by the imposition of suitable force on said first layer and/or said substrate.

21. A method for the preparation of the device of either of claims 1 or 8, which method comprises:
a. preparing a suitable biomaterial as a three dimensional structure selected from sheets, strips, strands of indefinite length and fibers;
b. treating at least one outer surface of the biomaterial prepared in Step a. to form thereon at least one said surface for the reception of said first layer of cells;
c. recovering said treated biomaterial defining the said at least one surface of Step b.;
wherein said biomaterial film of Step c. is adapted to serve as substrate for said device.

22. A method for the preparation of a bioartificial composite useful for repair of tissues or organs in a host, said method comprising:
a. preparing a substrate defining a surface having the morphological characteristics of the desired tissue;
b. applying to the surface of said substrate a cellular preparation, said cellular preparation comprising a quantity of cells capable of growth and aggregation to form a component of said tissue; and
c. implanting the bioartificial composite of Step b. at the location of desired repair,
whereby the growth of said tissue takes place in the host.

23. The method of claim 22 wherein said cellular preparation of Step b. is of a different cell type from that of said tissue.

24. The method of claim 22 wherein said cellular preparation of Step b. is genetically modified to deliver a therapeutic compound useful in the treatment of disease or the promotion of tissue repair.

25. A method for the repair of damaged tissues or organs in a mammal comprising preparing a biological composite in accordance with the method of claim 22.

26. A method for the preparation of tissue useful for repair of tissues or organs in a host, said method comprising:
a. preparing a substrate defining a surface having the morphological characteristics of the desired tissue;
b. applying to the surface of Step a. cellular preparation, said cellular preparation comprising a quantity of cells capable of growth and aggregation to form said tissue;
c. incubating the substrate of Step b. under conditions promoting the growth of said tissue thereon; and
d. recovering the tissue prepared in Step c.

27. A method for the preparation of tissue useful for testing, development and discovery, said method comprising:
a. preparing a substrate defining a surface having the following characteristics:
i. at least one cell accepting surface defined by an oriented surface roughness of at least 200 nm root mean squared;
ii. at least one cell accepting surface defined by a surface curvature of equal or greater than 0.016 microns⁻¹; and
iii. said substrate defines a repeating surface structure;
b. applying to the surface of Step a. a cellular preparation, said cellular preparation comprising a quantity of cells capable of growth and aggregation to form a layer of cells;
c. incubating the bioartificial product of Step b. with a different type of cell to effect growth of said tissue thereon; and
d. recovering the tissue prepared in Step c.;
wherein said tissue may be used as as a benchtop testing system or tissue surrogate.

28. A method for the preparation of tissue useful for repair of tissues or organs in a host, said method comprising:
a. preparing a substrate defining a surface having the following characteristics:
i. at least one cell accepting surface defined by an oriented surface roughness of at least 200 nm root mean squared;
ii. at least one cell accepting surface defined by a surface curvature of equal or greater than 0.016 microns⁻¹; and
iii. said substrate defines a repeating surface structure;
b. applying to the surface of Step a. a cellular preparation, said cellular preparation comprising a quantity of cells capable of growth and aggregation to a layer of cells;
c. incubating the bioartificial product of Step b. with a different type of cell to affect growth of said tissue thereon; and
d. recovering the tissue prepared in Step c;
wherein said tissue may be used for therapeutic purposes.

29. The method of claim 28 wherein said cellular preparation of Step b. is genetically modified to deliver a therapeutic compound useful in the treatment of disease or the promotion of tissue repair.

30. A method for the preparation of tissue useful for repair of tissues or organs in a host, said method comprising:
a. preparing said tissue as in claim 29; and
b. implanting said tissue at the desired site for tissue or organ repair.

31. The device of claim 1 wherein said cellular preparation comprises cells taken from said host.

32. The device of claim 8 wherein said cellular preparation comprises cells from the tissues adjacent to the site of the implant of the device, that grow along said platform surface.

33. The device of either of claims 1 or 8 wherein said substrate is bioresorbable.

34. The device of either of claims 1 or 8 wherein said substrate is flexible.

35. The device of either of claims 1 or 8 wherein said cells comprise cells of the nervous system.

36. The device of claim 35 wherein said cells are derived from the CNS.

37. The device of claim 35 wherein said cells are selected from neurons, glial cells, astrocytes, microglial cells, dorsal root ganglion (DRG) cells.
